(19) United States
(12) Patent Application Publication
(10) Pub. No.: US 2023/0295048 A1
(43) Pub. Date: Sep. 21, 2023

(54) METAL OXIDE CERAMIC MATERIAL, PRECURSORS, PREPARATION AND USE THEREOF

(71) Applicant: MATHYM, CHAMPAGNE-AU-MONT-D'OR (FR)

(72) Inventors: Erik CAMPOSILVAN, Lyon (FR); Omid AKHLAGHI, Lyon (FR); Vincent GARNIER, Oullins (FR); Yves JORAND, Lyon (FR); Jérôme CHEVALIER, Rillieux La Pape (US)

(21) Appl. No.: 18/017,258

(22) PCT Filed: Jul. 23, 2021
(86) PCT No.: PCT/EP2021/070739

§ 371 Date: Jan. 20, 2023

(30) Foreign Application Priority Data
Jul. 23, 2020 (FR) FR2007734

(51) Int. Cl. C04B 35/488; A61C 13/00; C04B 35/638; C04B 35/64; C04B 35/626; A61K 6/818; A61K 6/17

(52) U.S. Cl. CPC C04B 35/488; A61C 13/0022; C04B 35/638; C04B 35/64; C04B 35/6266; A61K 6/818; A61K 6/17; A61C 2201/002; C04B 2235/3246; C04B 2235/3229; C04B 2235/5454; C04B 2235/6027; C04B 2235/604; C04B 2235/606; C04B 2235/656; C04B 2235/661; C04B 2235/775; C04B 2235/608; C04B 2235/9661; C04B 2235/785; B28B 1/265

(57) ABSTRACT

The present invention relates to a green body, a pre-ceramic body and a ceramic body based on metal oxide particles, in particular zirconium oxide. The present invention also relates to the method of producing said materials and to the use thereof, in particular in the field of dentistry.

### FIELD OF THE INVENTION

The present invention relates to the synthesis of metal oxide ceramic materials, e.g., zirconia ceramics (zirconium oxide, ZrO₂). These ceramic materials may, in particular, be used in the field of dentistry.

### PRIOR ART

Depending on the intended fields of application, ceramics must have mechanical (e.g., bending strength) and optical (e.g., transmittance) properties, which are important to a greater or lesser extent.

Thus, in the field of dentistry, a high transmittance within the visible range is generally required for aesthetic reasons.

Even though many materials are available, there is still a need to improve their properties.

Zirconium oxide, or zirconia is one of the most widely used materials in the field of technical ceramics and for dental applications. This material is generally marketed in the field of dentistry in the form of a pre-ceramic material compatible with machining methods such as CAD/CAM (computer-aided design and manufacture). After machining, the pre-ceramic material is sintered at a temperature generally between 1400° C. and 1600° C., necessary for complete densification, making it possible to obtain microcrystalline zirconia, with a grain size of more than 200 nm.

One of the strategies used in the prior art to improve the mechanical and optical properties and the hydrothermal stability of zirconia is to obtain a nanometric microstructure (grain size of less than 200 nm). This is known as nanocrystalline zirconia. Ideally, to achieve this objective, it is preferable to reduce the sintering temperature, while maintaining complete densification of the ceramic material after sintering.

However, the nanocrystalline materials of the prior art do not make it possible to combine the mechanical and optical properties when their thickness exceeds 3 mm to 4 mm. More or less significant cracks appear in these materials, whatever the manufacturing stage (green body, pre-ceramic or ceramic), and in some cases, the presence of residual porosity or densification gradients greatly limits the optical and mechanical properties.

Document U.S. Pat. No. 9,822,039 describes the formation and use of a gel of ZrO₂ particles to form a ceramic body. For this purpose, a gel is prepared by concentration by osmotic compression of a dispersion of particles. After a step of shaping by centrifugation, the gel is used to form a green body, then pre-ceramic and ceramic materials with a thickness of less than 5 mm. However, this document does not specify the conditions for demoulding the gel (after centrifugation) to form the green body. It does not specify that the demoulding and drying conditions are crucial in searching for pre-ceramic and ceramic materials with advantageous properties, in particular, pre-ceramic materials without cracks of more than 500 μm, having a thickness of at least 5 mm and compatible with conventional machining methods such as CAD/CAM (computer-aided design and manufacture).

The document US 2018/0072628 describes a microcrystalline material comprising colouring agents and therefore not having a nanometric microstructure.

Sasahara et al. (Development of Y-TZP Pre-Sintered Blocks for CAD-CAM Machining of Dental Prostheses, Materials Science Forum, Vols. 591-593, 2008, pages 712-716) have described the preparation of pre-ceramics by dry process, by pressurising a powder of particles (400 bar to 5000 bar) before pre-sintering it between 900° C. and 1100° C.

Amat et al. have described pre-ceramic materials (Preparation of pre-sintered zirconia blocks for dental restorations through colloidal dispersion and cold isostatic pressing, Ceramics International, 44, 2018, pages 6409-6416; Machinability of a newly developed pre-sintered zirconia block for dental crown applications, Materials Letters, 261, 2020, 126996). However, the conditions for preparing and drying a green body are not described, whereas they may determine the properties of pre-ceramic or ceramic material. Furthermore, these pre-ceramic materials require a sintering temperature of 1450° C. to 1600° C. to obtain a completely densified ceramic body, which does not have a nanometric microstructure. The hardness and mechanical strength of these pre-ceramic materials are not specified.

Document EP 2 692 311 describes a pre-ceramic body having a density of between 30% and 95%.

The present invention makes it possible to remedy this problem by providing a ceramic having properties of bending strength, transmittance, and optionally opalescence, which are all superior to those of conventional materials. Thus, the present invention makes it possible to obtain nanocrystalline ceramics which are free of cracks, even when they are produced from materials having a thickness of more than 5 mm. The Applicant has developed a method that guarantees the absence of cracks and complete densification at reasonably low pre-sintering and sintering temperatures.

### SUMMARY OF THE INVENTION

The present invention relates to 1) a green body (P1, P1z) and its preparation method (PC1), 2) a pre-ceramic body (P2, P2z) and its preparation method (PC2), and 3) a ceramic body (P3, P3z) and its preparation method (PC3).

In general, the ceramic body is obtained by heat treatment of the pre-ceramic body, previously prepared from the green body. As indicated below, these materials (P1, P1z, P2, P2z, P3 and P3z) are free of cracks.

The properties of the ceramic body (P3, P3z) result from the initial conditions of preparing the green body (P1, P1z) and more precisely, as indicated below (PC1), from the highly crystalline nature (not amorphous) and from the size of the metal oxide particles (≤40 nm), from moulding by pressure filtration to form a wet body, from demoulding (relative humidity >80%) and from drying (relative humidity ≥90%) to form the green body (P1. P1z). In the case of a green body with a thickness of at least 5 mm, the initial conditions making it possible to improve the properties of the ceramic body (P3, P3z) advantageously include pressing (b1′) and/or step (b3) described below.

On the other hand, the methods PC1, PC2 and PC3 are particularly advantageous. The PC3 method makes it possible to obtain a ceramic material having a nanometric microstructure (grain size of less than 200 nm) under conditions that are less restrictive than those of the prior art. For example, the PC3 method does not require handling gels or performing centrifugation or supercritical drying.

Body P1z corresponds to body P1 when the latter is based on zirconium oxide and has specific characteristics highlighted below. The same applies to bodies P2z and P3z relative to P2 and P3, respectively. Thus, P1, P2 and P3 cover the particular embodiments P1z, P2z and P3z. Thus, bodies P1, P2 and P3 may correspond to bodies based on zirconium oxide, which do not correspond to the P1z, P2z and P3z. The method PC1 makes it possible to prepare body P1 and thus body P1z. The method PC2 makes it possible to prepare body P2 and thus body P2z. The method PC3 makes it possible to prepare body P3 and thus body P3z. Unless otherwise indicated, the characteristics of the methods relate to all bodies: P1 and P1z for PC1, P2 and P2z for PC2, P3 and P3z for PC3.

Unless indicated otherwise, the hardness of the various bodies corresponds to the Vickers hardness HV1. It is measured in accordance with the standard ISO 6507, with an indenter in the form of a standardised pyramid made of diamond with a square base and an angle at the apex between faces equal to 1360 to which a force is applied for a loading time of 10 to 15 seconds (if not specified otherwise). For HV1 measurements, the force applied is 1 kgf (1 kilogram-force, i.e., 9.80665 N or 9.80665 kg·m·s⁻²). The hardness may be expressed in Vickers units (kgf/mm²) or in International System units, e.g., in MPa (N/mm²).

Unless otherwise indicated, the mechanical biaxial bending strength of the various bodies is measured according to the standard ISO 6872:2015 with the “piston-on-three-ball strength tests” method and the indications provided for ceramics of type ii, class 1. The diameter of the support ring is 10 mm and the discs used for the measurement have a diameter of between 12 mm and 16 mm, and a thickness of 1.2 mm±0.2 mm. The mechanical biaxial bending strength corresponds to the mean of the measurements performed for 10 samples.

Green Body P1z

According to the invention, the green body has the particular feature of being a ceramic precursor having mechanical strength properties and optical properties (high opalescence and transmittance) that are particular sought after in the field of dentistry.

Furthermore, it makes it possible to form pre-ceramic bodies having a thickness of more than or equal to 5 mm, advantageously of more than or equal to 10 mm and more advantageously of more than or equal to 20 mm.

More precisely, green body P1z according to the invention comprises:
crystalline zirconium oxide particles having a mean size (by number) of between 3 nm and 25 nm, advantageously of between 4 nm and 15 nm, more advantageously of between 4 nm and 12 nm.
pores having a mean size of between 2 nm and 6 nm, more advantageously of between 3 nm and 5 nm.

On the other hand, green body P1z has:
an apparent density of between 45% and 60% relative to the theoretical density, advantageously of between 46% and 57%, and more advantageously of between 47% and 55%;
a thickness of more than or equal to 5 mm, advantageously of between 5 mm and 40 mm, more advantageously of between 10 mm and 40 mm and even more advantageously of between 20 mm and 40 mm.

The mean size of the pores (bodies P1, P1 z, P2, P2z) may be measured by conventional techniques, e.g., by bet (Brunauer-Emmett-Teller) analysis of the specific surface area of the body concerned (P1, P1z, P2, P2z) and by applying the BJH (Barrett-Joyner-Halenda) method to determine the size distribution.

The density (bodies P1, P1z, P2, P2z, P3, P3z) may be measured by conventional techniques, e.g., the Archimedes thrust method after weighing the body in air at 20° C. and suspended in deionised water at 20° C.

When the body concerned has an open porosity (bodies P1, P1z, P2, P2z), a thin layer of water-resistant acrylic polymer is deposited on the entire surface of the body before being weighed. In this case, it is an apparent density.

Furthermore, when the body concerned (bodies P1, P1z, P2, P2z, P3, P3z) has a regular and measurable geometry (e.g., a cube or a cylinder resulting from demoulding or shaping), the density and the apparent density may also be obtained by the simple mass/volume ratio. In this case, the volume is calculated from dimensional measurements and the mass is obtained by air weighing at 20° C.

The density (bodies P1, P1z, P2, P2z, P3, P3z) may be expressed as a percentage relative to the theoretical density in the absence of pores. The theoretical density depends on the dopant content and may be calculated by analysing X-ray diffraction patterns of the sintered bodies by the Rietveld method. This method makes it possible to estimate the lattice parameter of the phases present in the crystal lattice (quadratic and cubic phases in the case, according to the invention, of ZrO₂) for each composition. The theoretical density is then calculated as the ratio between the weight of the atoms present in the lattice (depending on the composition) and the volume of the lattice.

Advantageously, green body P1z comprises at least 85% by weight of zirconium oxide particles, more advantageously at least 90% by weight, and even more advantageously between 95% and 98% by weight, relative to the weight of the green body. These percentages by weight of ZrO₂ include the possible presence of dopant. This is the percentage by weight of the particles, whether or not they are doped.

Thus, green body P1z may comprise less than 10%, advantageously less than 5%, by weight of material(s) distinct from the zirconium oxide particles. It may be organic residues and/or water, e.g., binder or dispersant residues (see method PC1 below). Advantageously, green body P1z consists of zirconium oxide particles, doped or not doped.

The green body (P1 or P1z) is generally three-dimensional in shape. Thus, its thickness corresponds to its size, which is generally the smallest. The thickness may correspond to the height of the green body, in particular, when the latter is in the form of a block or of a disc.

In general, the green bodies of the prior art consisting of particles, in particular, ZrO₂, with a size of less than 40 nm are relatively fine insofar as the methods of the prior art do not make it possible to obtain a green body free of cracks and at least 5 mm thick.

The thickness of green body P1z is more than or equal to 5 mm, advantageously more than or equal to 10 mm. In general, it is less than or equal to 40 mm.

The other two main dimensions (or the diameter) of green body P1z are advantageously, and independently of each other, more than or equal to 5 mm and even more advantageously more than or equal to 20 mm. They are generally less than or equal to 150 mm.

Thus, green body P1z may be in the form of a disc 150 or 100 mm in diameter and 40 mm thick. For example, it may also be a block with the dimensions 100 mm×100 mm×40 mm.

The zirconium oxide particles may be doped, in particular, with a metal oxide such as yttrium oxide (yttria, Y₂O₃) or cerium oxide (ceria, CeO₂). According to another embodiment, the dopant may be magnesium oxide (MgO), calcium oxide (CaO), gadolinium oxide (Gd₂O₃), scandium oxide (Sc₂O₃), or niobium oxide (Nb₂O₅). The particles may also be doped with a mixture of several oxides.

The dopant advantageously represents 1 mol % to 15 mol % relative to the total number of moles of ZrO₂, more advantageously 1 mol % to 10 mol %, and even more advantageously 1 mol % to 12 mol %. It is the maximum quantity of dopant(s) even when the particles contain, e.g., two types of dopants.

For example, the quantity of dopant(s) may be between 1 mol % and 2.5 mol %, or between 2.5 mol % and 3.5 mol %, or between 3.5 mol % and 4.5 mol %, or between 4.5 mol % and 6.5 mol %, or between 6.5 mol % and 12 mol %.

Thus, according to a preferred embodiment, the crystalline metal oxide particles are zirconium oxide particles, advantageously doped with 1 mol % to 15 mol %, more advantageously 1.5 mol % to 13.5 mol %, of a dopant advantageously chosen from yttrium oxide and cerium oxide.

Thus, the zirconium oxide is advantageously doped with 1.0 mol % to 15 mol % (advantageously 2.5 mol % to 10.5 mol %) of yttrium oxide or with 5.0 mol % to 15 mol % of cerium oxide.

In general, and because of its production method, zirconium oxide may also contain a reduced quantity of impurities, typically less than 5 mol %, of hafnium oxide (HfO₂). The presence of hafnium oxide is inherent in producing zirconium oxide and remains difficult to separate from the zirconium oxide.

It is preferable that the green body (and therefore the dispersion in step (a1) below) comprise only one type of crystalline metal oxide particles. However, a mixture of crystalline metal oxide particles and their doped equivalents may be used. For example, a mixture of ZrO₂ particles and Y₂O₃ doped ZrO₂ particles may be used. It may also be a mixture of Y₂O₃ doped ZrO₂ particles and CeO₂ doped ZrO₂ particles.

The green body (P1 or P1z) is advantageously free of cracks. According to the invention, a material free of any number of cracks does not comprise any maximum number of cracks of more than 500 μm in size. More advantageously, it does not comprise of any number of cracks of more than 50 μm, even more advantageously of more than 30 μm. The crack size may be measured, e.g., on a polished surface or by fractography after the body has been stressed, e.g., in bending, up to a load causing it to rupture. Observing the polished surface or of the fracture surface by optical or electron microscopy generally makes it possible to identify cracks or defects and to measure their size. Penetrating liquids may be used to reveal cracks for optical microscopy observations.

Green body P1z is generally used to form a pre-ceramic body P2z or a ceramic body P3z.

Method PC1 for Preparing Green Body P1

Green body P1 may be produced according to the method PC1 which may also be followed to prepare green bodies from particles of metal oxides other than ZrO₂.

The method PC1 for preparing a green body P1 comprises the following steps:
(a1) providing a dispersion of crystalline metal oxide particles having a mean (number) size of 40 nm or less;
(b1) introducing the dispersion into a mould and forming a wet body by pressure filtration of the dispersion in the mould;
(b1′) optionally, pressing the wet body by pressure filtration;
(b2) demoulding the wet body under conditions of relative humidity of more than 80%;
(b3) optionally, when the wet body has a density gradient, removing the part of the wet body at the end of filtration;
(c1) forming a green body P1 by drying the wet body under conditions of relative humidity of more than or equal to 90%,
(c1′) optionally, shaping the green body P1.

Step (a1)

The dispersion in step (a1) is a dispersion of particles in a solvent. This solvent is advantageously chosen from the group comprising water and alcohols, in particular, isopropanol. They are preferably a dispersion in a protic solvent, advantageously water.

The use of a powder of particles having a mean size (by number) of less than or equal to 40 nm instead of the dispersion does not make it possible to perform the steps of forming a wet body (b1) and demoulding (b2). Furthermore, the formation, from a powder, of a pre-ceramic material requires, when the particles are made of ZrO₂, a pre-sintering temperature of 900° C. to 1100° C. and a sintering temperature of 1300° C. to 1600° C., making it impossible to obtain a nanometric microstructure (grain size of less than 200 nm) in the ceramic body.

The pH of the dispersion may be acidic or basic. However, according to a preferred embodiment, the pH of the dispersion is between 7 and 14, advantageously between 7 and 11.

The crystalline metal oxide particles are advantageously homogeneous in terms of shape and size. In other words, it is preferable that at least 90% by volume of the particles have the same shape, more advantageously at least 95%, and even more advantageously 100%. Furthermore, it is preferable for at least 90% by volume of the particles to have a size identical to plus or minus 6 nm, more advantageously at least 95%, and even more advantageously 100%.

The crystalline metal oxide particles may, in particular, be spherical, cylindrical, cubic or rod-shaped. Advantageously, they are either spherical or approximately spherical in shape.

The metal oxide particles may also be functionalised according to the general technical knowledge of the person skilled in the art.

The crystalline metal oxide particles have a mean size (by number) of less than or equal to 40 nm, preferably of between 1 nm and 40 nm, advantageously of between 2 nm and 30 nm, more advantageously of between 3 nm and 25 nm, even more advantageously of between 4 nm and 15 nm, and even more advantageously of between 4 nm and 12 nm. In the case of ZrO₂ particles, the mean size (by number) is advantageously between 3 nm and 25 nm.

The term “size” means the largest dimension of the particles, e.g., the diameter for spherical particles or the length for cylindrical or rod-shaped particles. It is the mean size of at least 300 particles which may, in particular, be measured, in a conventional manner, by image analysis from transmission electron microscopy (TEM) images.

The crystalline metal oxide particles may form agglomerates in the dispersion, whose size is advantageously less than 50 nm, more advantageously between 14 nm and 30 nm. Beyond 50 nm, the agglomerates may generate the presence of residual pores having a mean size of more than 20 nm in green body P1 that cannot be removed during the formation of a ceramic material (step (f1) below).

The dispersion may also comprise at least one binding agent. This binding agent is advantageously chosen from the group comprising PEG (polyethylene glycol), polymers based on acrylate and/or methacrylate, PVA (polyvinyl alcohol) and PVP (polyvinyl pyrrolidone), and mixtures thereof. Polyols of lower molecular weight than PEG or PVA, e.g., glycerol, may be added as a binding agent and/or to modify the characteristics of the binder. Thus, the binding agent may, in particular, be advantageously chosen from the group comprising PVP; PVA; mixtures of PVP and of PEG; and mixtures of PVA and of glycerol.

The binding agent advantageously represents 1.5% by weight or less, e.g., between 0.4% and 1.5% by weight, more advantageously 0.4% to 1.2% by weight, and even more advantageously 0.5% to 1% by weight, relative to the weight of the particles contained in the dispersion in step (a1).

The binder makes it possible to increase the strength of the green body and thus to reduce the formation of cracks while the green body is being dried according to step (c1).

The person skilled in the art will be able to adjust the quantity of binder so that the viscosity of the dispersion is unaffected by its presence. Furthermore, the binder is removed during the debinding operation (d1).

The incorporation of binder into the dispersion of metal oxide particles is generally followed by a stirring step, e.g., by mechanical stirring and/or sonication. This step subsequently makes it possible, while the green body is being prepared, to ensure the homogeneity of the green body. Furthermore, the absence of stirring via sonication generally results in the appearance of cracks during the drying and/or debinding steps, or the appearance of pores of mean size of more than 20 nm in ceramic body P3.

Advantageously, the dispersion in step (a1) comprises a dispersing agent. This dispersing agent may, in particular, be chosen from the group comprising linear or non-linear molecules (including polymers and oligomers) and having at least one functional group capable of forming a bond or a strong interaction with the surface of the particles, e.g., a polar functional group, e.g., of the C(O)OH carboxylic acid type. The dispersing agent may, in particular, be chosen from the group comprising carboxylic acids, amino carboxylic acids, glycolic acids and ethoxylated carboxylic acids, poly(acrylic acid) and poly(methacrylic acid) acids and their salts. These compounds may be linear or branched. The carboxylic acids and the carboxylic amino acids advantageously have a number of carbons of between 1 and 10, advantageously of between 2 and 10.

Advantageously, the dispersing agent has a molecular weight of between 60 g/mol and 360 g/mol, more advantageously of between 130 g/mol and 260 g/mol.

The presence of an excessive quantity of dispersant may reduce the rate of formation of the wet body during step (d1) and even in some cases stop filtration. Thus, the dispersing agent advantageously represents 0.5% to 8% by weight, more advantageously 1% to 4.5% by weight, relative to the weight of the dispersion in step (a1).

The stability of the dispersion is an important parameter so as to avoid the modifying the dispersion during filtration step (b1) and thus prevent the formation of flocculated or agglomerated particles or a non-homogeneous dispersion.

The presence of a dispersing agent makes it possible to stabilise the dispersion, giving a colloidal dispersion. It also makes it possible to reduce the viscosity of the dispersion. Thus, it is possible to increase the concentration of metal oxide particles by reducing the weight ratio of the solvent to the crystalline metal oxide particles. Consequently, it is easier to form the dispersion in the presence of a dispersing agent.

The dispersing agent may, in particular, be chosen from the groups of di-carboxylic and tri-carboxylic acids, comprising, in particular, triammonium citrate (TAC, C₆H₁₇N₃O₇), dibasic ammonium citrate (DAC, C₆H₁₄N₂O₇), citric acid (C₆H₈O₇), tartaric acid (C₄H₆O₆), malic acid (C₄H₆O₅), from the group of ethoxylated carboxylic acids comprising, in particular, 2-[2-(2-methoxyethoxy)ethoxy]acetic acid (MEEEA), 2-(2-methoxyethoxy) acetic acid (MEEA), and from the group of poly(acrylic acid) and poly(methacrylic acid) (150 to 360 g/mol), and mixtures thereof.

The crystalline metal oxide particles of the dispersion in step (a1) are advantageously selected from the group comprising ZrO₂, Al₂O₃, CeO₂, MgO, YAG (yttrium aluminium garnet of formula Y₃Al₂(AlO₄)₃), MgAl₂O₄, AlON, Mullite (aluminium silicate, e.g., of formula 3Al₂O₃, 2SiO₂), Y₂O₃, Sc₂O₃, Lu₂O₃, and mixtures thereof.

They are advantageously ZrO₂ particles, in particular, for ceramics for dental application.

These particles may be doped, in particular, with a metal oxide that is different from the material constituting the majority of the crystalline particles. In the case of zirconium oxide, the dopant advantageously represents 1 mol % to 15 mol % relative to the entire quantity of the crystalline metal oxide particles, more advantageously 1.5 mol % to 13.5 mol %. The particular embodiments described for the zirconium oxide particles are also applicable to the other metal oxides.

According to a particular embodiment, bodies P1, P2 and P3 (advantageously P1z, P2z and P3z) may have a lanthanum oxide concentration of less than 0.1 mol % relative to the total number of moles of metal oxide. Thus, advantageously, the dopant is not lanthanum.

According to a preferred embodiment, the crystalline metal oxide particles are zirconium oxide particles, advantageously doped with 1 mol % to 15 mol % of a dopant advantageously chosen from yttrium oxide and cerium oxide.

It is preferable that the dispersion comprise only one type of crystalline metal oxide particles. However, a mixture of crystalline metal oxide particles and their doped equivalents may be used. For example, a mixture of ZrO₂ particles and Y₂O₃ doped ZrO₂ particles may be used.

Furthermore, according to a particular embodiment, the dispersion may comprise an additive which is different from the binding agent and from the dispersing agent, e.g., a dye or a sintering additive. The dye and sintering additive are advantageously in the form of particles or precursors of at least one other type of metal oxide, e.g., iron oxide particles as dye and aluminium oxide particles as sintering additive. The dye may, in particular, be chosen from the particles or particle precursors of the oxides of the elements d and f in the periodic table of the elements. For example, the oxides of the elements Pr, Er, Fe, Co, Ni, Ti, V, Cr, Cu, Mn, Tb or Ce may be used as colouring agents which are useful in the field of dentistry. Each metal oxide, used as an additive, advantageously represents between 0.002% by weight (20 ppm) and 1.5% by weight relative to all of the metal oxides.

The dye may be introduced initially into the dispersion of crystalline metal oxide particles or during filtration (b1). In both cases, it is possible to obtain a colour gradient over the thickness of body P1 and therefore of bodies P2 and P3 by controlling the concentration of the dye in the dispersion and its tendency to sediment or migrate during filtration.

Zirconium dioxide may be in crystalline form and have three different crystal structures depending on the temperature, presence of dopants and size of the crystals: monoclinic, quadratic (tetragonal) and cubic. For particles of nanometric size, it is difficult to distinguish the quadratic and cubic phases by the X-ray diffraction (XRD) measurement method. They are therefore generally labelled together under the name “quadratic/cubic”. The monoclinic phase is easily distinguishable from the other two.

The metal oxide particles are crystalline. When it is in the form of ZrO₂ particles, doped or not, the quadratic/cubic crystalline form is preferred. Advantageously, at least 50% of the ZrO₂ particles, doped or not, are in quadratic/cubic crystalline form, more advantageously at least 70%, even more advantageously at least 90%, and even more advantageously at least 99%, relative to the volume of the ZrO₂ particles, doped or not.

The particles of ZrO₂, doped or not, may be prepared according to conventional techniques, e.g., according to one of the methods described in patent application FR 1872183 and patent U.S. Pat. No. 8,337,788.

The crystalline metal oxide particles advantageously represent 10% to 50% by weight, more advantageously 20% to 40% by weight, relative to the weight of the dispersion in step (a1). The weight of the crystalline metal oxide particles includes the weight of the optional dopant and optional dye.

The concentration of crystalline metal oxide particles may be adjusted before step (b1), in particular, by the conventional techniques tangential filtration, controlled evaporation and osmotic compression.

A concentration of metal oxide particles of between 10% and 50% by weight generally makes it possible to ensure rapid filtration while maintaining a relatively low viscosity, advantageously less than 1 Pa·s, preferably less than 100 mPa·s at 25° C.

In general, when the concentration of metal oxide particles is more than 50% by weight, the green body obtained after drying step (c1) has cracks. Dispersions having a metal oxide particle concentration that is too low (<10%) require a long filtration time (e.g., at least 5 days) to obtain a satisfactory green body thickness (5 mm or more).

Prior to filtration step (b1), the dispersion may be degassed. This step makes it possible, in particular, to remove any gases dissolved in the dispersion. Performing this step may improve the quality of the pre-ceramic and ceramic bodies.

Step (b1)

Advantageously, the filtration in step (b1) is performed via filtration by applying pressure to the filtered dispersion. The pressure exerted is advantageously between 5 bar and 50 bar, more advantageously of between 10 bar to 40 bar, and even more advantageously between 12 bar and 30 bar. The person skilled in the art will be able to adjust this pressure according to the filtration rate of the dispersion used so as to optimise the density of the wet body and thus reduce the density gradients, i.e., the local concentration of metal oxide particles. According to a particular embodiment, the filtration in step (b1) may be performed by applying pressure to the dispersion by means of a fluid, e.g., a gas or a liquid. According to another embodiment, it may be performed by applying a mechanical force to the dispersion, e.g., by means of a piston.

If too low a pressure is applied, e.g., between 1 and 3 bar, it is possible that the filtration speed is too low to form a wet body that has a satisfactory thickness and is free of cracks. Furthermore, the density of the wet body may, in this case, not be sufficient to obtain a pre-ceramic (P2, P2z) and/or ceramic (P3, P3z) material that is sufficiently dense for a given heat treatment temperature, requiring higher pre-sintering and sintering temperatures. On the other hand, if the pressure applied is too high, e.g., between 60 and 80 bar, it is possible to form large density gradients or to cause the formation of large residual stresses in the wet body, which would result in the cracks being formed during demoulding, while drying the wet body and/or while forming the ceramic material.

The filtration is advantageously performed by applying a pressure by means of a fluid or a piston. Typically, the pressure is increased progressively to reach a certain value (advantageously 5 bar to 50 bar). Advantageously, the pressure increase lasts for between 5 minutes and 20 minutes. Next, filtration continues at a constant pressure (advantageously 5 bar to 50 bar) for a period which may vary from a few minutes to a few hours, advantageously from 10 minutes to 100 hours, more advantageously from 60 minutes to 48 hours. During filtration, the thickness of the wet body gradually increases. The person skilled in the art will be able to adjust the pressure and duration so as to obtain a wet body with the thickness required.

The end of filtration is advantageously achieved by reducing the pressure. The progressive reduction of the pressure exerted on the dispersion advantageously lasts between 3 minutes and 100 minutes, more advantageously between 5 minutes and 30 minutes. This operation makes it possible to release the elastic energy stored in the wet body while controlling the possible formation of internal stresses. Without checking this operation, cracks may be formed before or during drying according to step (c1). The drop in pressure makes it possible to pass progressively (advantageously 3 minutes to 100 minutes) from a high pressure (advantageously 5 bar to 50 bar) to atmospheric pressure, of approximately 1 bar.

When a dye or its precursor is added to the dispersion, its concentration in the dispersion, while the wet body is being formed, may advantageously vary over time. In fact, all or part of the dye may have a colloidal stability lower than that of the crystalline metal oxide particles, and may therefore partially or completely sediment before the end of filtration step. In this case, the wet body may have a concentration gradient of the dye. This dye gradient may advantageously be controlled, e.g., by adjusting the stability of the dye in the dispersion and its tendency to settle. If the dye is magnetic, the application of a magnetic field may advantageously make it possible to control the settling rate or to cause the dye to migrate to obtain the desired colour gradient. This embodiment makes it possible to prepare a body (pre-ceramic P2 or ceramic P3) having a colour gradient.

Thus, bodies P1, P2 and P3 (including P1z, P2z and P3z) may have a concentration gradient of a colouring agent or colouring agent precursor.

Optional Step (b1′)

This step consists of pressing the wet body resulting from filtration step (b1). It may be performed at the end of a partial or total filtration of the dispersion during step (b1). It is particularly suitable for preparing green bodies having a thickness of at least 5 mm, e.g., P1z.

More precisely, after step (b1) and before the demoulding step (b2), a pressing step may be performed so as to homogenise, if necessary, the wet body and thus reduce any density gradients. This pressing (b1′), advantageously performed with a pressure of between 10 bar and 50 bar (1 bar=10⁵ Pa), also makes it possible to avoid cracks being formed in the green body having a thickness of at least 5 mm, e.g., P1 z. This is, in particular, the reason why this step is particularly suitable if a green body is at least 5 mm thick. For this purpose, it is possible to use a pressing (b1′) by means of a fluid or a piston, as already described for step (b1).

When pressing is performed by means of a fluid, the unfiltered dispersion during filtration step (b1) is discharged and a second dispersion may be used for step (b′), advantageously a second dispersion having a particle concentration lower than that in step (b1), the particles being of identical nature and advantageously of identical or smaller size. The rate of formation of the wet body during step (b1′) is advantageously lower than that in step (b1). The person skilled in the art will be able to adjust the filtration pressure (b1′) or the composition of the dispersion to control the pressing/filtration rate. In this case, the dispersion is advantageously partially filtered during step (b1). It is thus possible to remove the presence of density gradients in the wet body formed during step (b1).

In the case where pressing is performed by means of a fluid, the method advantageously comprises step (b3) making it possible to remove the part of the wet body formed from the second dispersion.

When pressing is performed by means of a piston or another mechanical force, pressing (b1′) is advantageously performed directly by piston action on the wet body in step (b1). In this case, the entire dispersion is advantageously filtered during step (b1) and step (b1′). Thus, by carrying out a second pressing (b1′), it is possible to remove the presence of density gradients in the entire green body. In this case, the optional step (b3) is advantageously not performed.

When the pressure used in step (b1′) is similar, or identical, to the pressure used in step (b1), this optional pressurisation step is advantageously performed for a period of between 0.1 times and 0.5 times the duration of the first filtration step.

A pressing b1′ that is too long generally leads to cracking of the wet body before demoulding, whereas a pressing b1′ that is too short generally does not make it possible to completely remove the density gradient. Also, the person skilled in the art will be able to adjust the pressing time (b1′) depending on the pressure used.

Advantageously, steps (b1) and (b1′) are performed with the same fluid or mechanical means.

Step (b2)

Demoulding in step (b2) is performed in an environment having a relative humidity of more than 80%, preferably more than 90%. These conditions make it possible to optimise the formation of a green body free of cracks. They also contribute to avoid cracks being formed in the wet body when it is being demoulded.

No particular technique is required to perform this step. On the other hand, it is performed under special conditions in terms of relative humidity to avoid cracks being formed. In fact, below 80% relative humidity, the green body may have cracks, in particular, when it has a thickness of at least 5 mm. Demoulding below 80% relative humidity may also reduce the characteristics of the pre-ceramic bodies (P2, P2z) and/or of the ceramic bodies (P3, P3z), even if the green body is not cracked.

The outer surface of the wet body may advantageously be kept wet as soon as it is demoulded until step (c1) begins, e.g., by exposure to a flow of wet mist or by immersion in water or another solution, to avoid any uncontrolled evaporation from the surface of the wet body.

Step (b3)

Step (b3) is optional. It is generally performed when the wet body has a gradient in terms of density, i.e., in terms of local concentration of metal oxide particles. It is particularly suitable if a green body is at least 5 mm thick.

Step (b3) may replace or complete pressing (b1′). Thus, the method may comprise pressing step (b1′) and/or step (b3), in particular, when the green body has a thickness of at least 5 mm.

Optional step (b3) is advantageously performed at a relative humidity of at least 80%, preferably under the relative humidity conditions of demoulding in step (b2).

To reduce the possible residual density gradient, it is possible to remove the part of the wet body that has been formed at the end and, optionally, the part formed at the start of filtration step (b1) or of pressing step (b1′). In other words, in a vertical device having a means (piston or fluid) exerting pressure on the dispersion in an up and down movement, it is a question of removing the upper part and optionally the lower part of the wet body. This operation is generally implemented when the pressure means is a fluid, such as a gas. It may be useful and can be implemented by any means, e.g., a blade or a wet abrasive sponge. Thus, thanks to this operation, the remaining part of the wet body is not modified and the density gradient is virtually zero within the material.

Step (c1)

The wet body used during step (c1) advantageously consists of particles of metal oxide, a dispersant, a binder, optionally a dye and residual solvent(s). The density of this material generally depends on the pressure used during filtration and on the composition of the dispersion.

The relative humidity, during drying according to step (c1), is more than or equal to 90%, more advantageously of between 90% and 100%, and even more advantageously of between 95% and 100%. Advantageously, it is maintained under these conditions for at least a few hours, more advantageously for 24 hours.

In fact, below 90% relative humidity, the green body may have cracks, in particular, when it is at least 5 mm thick. Demoulding below 90% relative humidity may also reduce the characteristics of the pre-ceramic bodies and/or ceramic bodies, even if the green body is not cracked. In this case, the pre-ceramic bodies and/or ceramic bodies may require respectively higher pre-sintering and sintering temperatures to achieve higher densification, which generates a coarser (non-nanometric) microstructure in the ceramic body or the presence of a large degree of residual porosity. Residual porosity of large size (>20 nm) progressively alters the optical properties of the ceramic body, as porosity increases.

Between steps (b2) and (c1), the wet body is advantageously maintained at a relative humidity of at least 80%, preferably during optional step (b3).

Step (c1) may be performed by means of any device, e.g., in a chamber with a controlled humidity. In particular, it may be a device in which the wet body is exposed to a relative humidity that decreases progressively and in a controlled manner, e.g., 24 hours at a relative humidity of more than or equal to 90%, and then decreases by 10% every 24 hours up to 40%.

Advantageously, the wet body is positioned on a substrate, e.g., a grid, making it possible to expose all parts of the wet body. Thus, drying is homogeneous and makes it possible to avoid or to reduce the formation of cracks.

Furthermore, the lower the density gradient of the wet body, the greater the probability of obtaining a green body free of cracks. This technical effect is optimised thanks to a relatively long drying time that can be adjusted depending on the thickness of the wet body. Thus, step (c1) may have a duration advantageously of between a few hours and a few days, more advantageously of between 1 day and 10 days, more advantageously of between 3 days and 7 days, e.g., of approximately 5 days. This duration includes maintaining the relative humidity at a minimum of 90% and decreasing it, advantageously to 40%. However, as already indicated, the relative humidity is advantageously maintained at 90% for several hours.

The drying temperature during step (c1) may advantageously be between 20° C. and 99° C., more advantageously between 20° C. and 60° C.

At the end of drying according to step (c1), the green body has residual pores having a mean size of between 2 nm and 6 nm, advantageously of between 3 nm and 5 nm. The use of a dispersion with good colloidal stability and homogeneity, combined with the pressure filtration technique, makes it possible to optimise the mean size of the residual pores.

The thickness of green body P1 (with P1*P1z) is not limited, it is advantageously at least 1 mm, and generally less than or equal to 40 mm.

The other two main dimensions (or the diameter) of the green body (P1 or P1z) are advantageously, and independently of each other, more than or equal to 5 mm. They are generally less than or equal to 150 mm.

Thus, the green body (P1 or P1z) may be in the form of a disc 150 mm or 100 mm in diameter and 40 mm thick. For example, it may also be a block with the dimensions 100 mm×100 mm×40 mm.

Step (c1′)

This step is optional. It makes it possible to shape green body P1 before any pre-sintering or sintering step.

It may be put back into operation by means of an abrasive tool, e.g., a disc or a pre-polishing grinding wheel (e.g., made of silicon carbide SiC or diamond, with a grain typically comprised, e.g., between a value of 100 and 1500), or an abrasive milling cutter for glass-ceramic which may be mounted on a single-axis or multi-axis milling machine. This step may be performed in the absence of water, or solvent(s) or lubricating fluid(s).

This step may replace or complete step (e2) described below.

Pre-Ceramic Body P2 or P2z

Pre-ceramic body P2 or P2z according to the invention is based on metal oxide, zirconium oxide for P2z. The pre-ceramic body P2z of zirconium oxide has:
a mean grain size of less than 45 nm, preferably of between 5 nm and 30 nm, advantageously of between 5 nm and 20 nm,
a density of between 52% and 68%, advantageously of between 53% and 63%, relative to the theoretical density,
a mean pore size of between 2 nm and 15 nm,
a hardness of more than 150 HV, advantageously of between 150 HV and 200 HV, and
a mechanical biaxial bending strength of at least 25 MPa, advantageously of between 25 MPa and 40 MPa.

As already indicated for P1 and P1z, the metal oxide may be doped.

The pre-ceramic body P2z is composed (advantageously consisting) of partially sintered zirconia oxide particles (grains), whose partial sintering forms necks (bridges) between the grains. The void volumes existing between the grains correspond to the pores.

The mean grain size (P2, P2z) may, in particular, be measured by transmission electron microscopy (TEM) image analysis. It may be performed on a fine plate prepared, e.g., by ionic polishing from a volume representative of the material P2 or P2z. The mean grain size (P2, P2z) may also be measured by the Scherrer method, from measuring the width of the main X-ray diffraction peaks at mid-height (full width at half maximum—FWHM) and this according to the conventional procedure, after having subtracted the Kα2 component from the diffraction spectrum and having corrected the FWHM measurement taking into account the widening of the peaks due to the device (widening of the peaks due to the instrument—“instrumental peak broadening”). In the case of P2z, the peaks selected for measuring are generally (−111) and (111) for the monoclinic phase and (111) for the quadratic/cubic phase. The mean grain size is then calculated using the Scherrer equation:

Mean size=(Kλ)/(β cos θ)

In this equation, K is the form factor (0.89), λ is the wavelength, β is the corrected FWHM value, and θ is half of the angle of the selected peak.

It is more precisely a mean size of the crystallites, which corresponds to the mean size of the grains if the grains are monocrystalline, as in the case of body P1, P1z, P2 or P2z.

The pre-ceramic body (P2 or P2z) has a mean pore size advantageously of less than 10 nm. The reaction temperature may, in particular, be between 4 nm and 9 nm, more advantageously between 5 nm and 8 nm.

The pre-ceramic body (P2 or P2z) is generally three-dimensional in shape. Thus, its thickness corresponds to its smallest size. The thickness may correspond to the height of the pre-ceramic body, in particular, when the latter has the shape of a block or of a disc.

The pre-ceramic body P2z advantageously has a thickness of more than or equal to 5 mm, more advantageously of more than or equal to 10 mm, and even more advantageously of more than or equal to 20 mm. In general, it is less than or equal to 40 mm.

The other two dimensions (or the diameter) of the pre-ceramic body P2z are advantageously, and independently of each other, more than or equal to 5 mm. They are generally less than or equal to 150 mm.

Thus, green body P2z may be in the form of a disc 150 or 100 mm in diameter and 40 mm thick. For example, it may also be a block with the dimensions 100 mm×100 mm×40 mm.

The three dimensions (or two [diameter+height] in the case of a cylindrical shape) of the pre-ceramic body (P2 or P2z) are generally less than those of the green body (P1 or P1z) because of a shrinkage phenomenon taking place during pre-sintering step (e1) described below. This linear shrinkage is generally of approximately 0% to 15%, more generally 1% to 10%, even more generally 2% to 9%, relative to at least one dimension of green body P1 or P1 z. Without putting forward any theory, the Applicant considers that the combination of the specific steps of pressure filtration (b1), advantageously pressing (b1′) and/or step (b3), demoulding (b2) (relative humidity >80%) and drying (c1) (relative humidity | 90%) make it possible to pre-sinter (e1) (400° C. to 800° C.) and sinter (f1) (900° C. to 1300° C., PC3 method below) under mild conditions. This set of conditions results in: (i) obtaining a pre-ceramic body (P2, P2z) adapted to conventional machining techniques, (ii) obtaining a nanocrystalline ceramic material (P3, P3z) with satisfactory mechanical and optical properties, and (iii) controlling the shrinkage phenomenon (0% to 15%) whereas, in the prior art, a shrinkage of almost 50% may be observed. Thus, thanks to the present invention, it is not necessary to oversize the green bodies (P1, P1z) and the pre-ceramic bodies (P2, P2z) to obtain a ceramic material (P3, P3z) having the desired dimensions.

P2z is advantageously a zirconium oxide pre-ceramic body doped with 1 mol % to 15 mol % of a dopant selected from yttrium oxide and cerium oxide.

Advantageously, the pre-ceramic body (P2 or P2z) is free of cracks. According to the invention, a material free of any number of cracks does not comprise any maximum number of cracks of more than 500 μm in size, more advantageously of more than 50 μm, even more advantageously of more than 30 μm.

The pre-ceramic body (P2 or P2z) has properties allowing it to be handled, transported and applied, e.g., by bonding, to a support compatible with a machine for machining, e.g., a multi-axis milling machine, without damaging it. Furthermore, its hardness makes it easily machinable. It is usually used to form a ceramic body (P3 or P3z). For example, when it takes the form of a block, it may be machined in the form of a dental prosthesis including, in particular, crowns, bridges, inlays or onlays. Once machined, the pre-ceramic body (P2 or P2z) can be sintered, in particular, according to step (f1) of the PC3 method described below.

Method PC2 for preparing the pre-ceramic body P2 Preparing ceramic body P2 involves the preliminary formation of green body P1 according to steps (a1) to (c1) of method PC1 (optionally (a1) to (c1′)). The pre-ceramic body P2 may be produced according to the method PC2 which may also be followed to prepare pre-ceramic bodies from metal oxide other than ZrO₂.

Thus, the method for preparing a pre-ceramic body P2 comprises the following steps:
(d1) optionally, debinding the green body formed according to method PC1;
(e1) forming a pre-ceramic body P2 by pre-sintering green body P1 of one of steps (c1), (c1′) or (d1), at a temperature of between 400° C. and 800° C., advantageously of between 480° C. and 700° C., more advantageously of between 500° C. and 650° C.

Step (d1)

During the optional debinding step (d1), any residual organic compounds are removed, typically by thermal decomposition. Debinding is promoted by the presence of pores within the green body, the organic compounds generally being removed in gaseous form. The presence of open pores in the green body and the use of a limited total amount of organic material thus make it possible to avoid any local stress which could subsequently generate cracks or defects in the pre-ceramic or ceramic material.

The debinding reaction is advantageously performed at a temperature of between 450° C. and 650° C., more advantageously of between 500° C. and 600° C.

Debinding is advantageously performed for a period of between 720 minutes and 21000 minutes; more advantageously of between 1440 minutes and 7200 minutes. This duration includes the time necessary to reach the debinding temperature, the permanence time at this temperature and the time necessary to reach the ambient temperature at the end of debinding.

Advantageously, debinding has a rise and a fall in temperature of between 0.05° C./min and 1° C./min, more advantageously of between 0.1° C./min and 0.5° C./min. The gradient rise and gradient fall are independent of each other.

These conditions make it possible to unbind, without any harmful effect, a green body more than 5 mm thick, that was not possible in the prior art. Thus, the green body does not have any cracks.

Step (e1)

Step (e1) is a pre-sintering step. In general, it is performed at a temperature of more than that of the debinding.

Pre-sintering is performed at a temperature advantageously of between 400° C. and 800° C., preferably of between 450° C. and 750° C.; more advantageously of between 480° C. and 700° C.

This pre-sintering temperature is lower than that of conventional materials. This provides an advantage given that a finer crystal size is retained within the pre-ceramic body. Furthermore, less expensive and energy-intensive furnaces may be used. The presence of small-sized pores (advantageously less than 15 nm on mean) within the pre-ceramic body and of necks (bridges) between the grains makes it possible to increase its hardness without negatively impacting its machining. It should be observed, however, that when pre-sintering is performed at more than 800° C., the machining generates vibrations which may possibly cause premature wear of the machining tools or cracking of the pre-ceramic body.

Pre-sintering is performed for a period advantageously of between 500 minutes and 10000 minutes; more advantageously of between 1000 and 7000 minutes. This duration includes the rise, permanence and fall time at the end of pre-sintering.

Advantageously, pre-sintering has a rise and a fall in temperature of between 0.05° C./min and 1° C./min, more advantageously of between 0.1° C./min and 0.5° C./min. The gradient rise and gradient fall are independent of each other.

In another particular implementation, the debinding and pre-sintering steps are performed simultaneously.

The pre-ceramic body P2 thus obtained may, in particular, serve as a precursor for preparing ceramic bodies P3 that can be used in the field of dentistry.

The thickness of pre-ceramic body P2 (with P2 #P2z) is not limited, it is advantageously at least 1 mm, and generally less than or equal to 40 mm.

The other two main dimensions (or the diameter) of the pre-ceramic body (P2 or P2z) are advantageously, and independently of each other, more than or equal to 5 mm. They are generally less than or equal to 150 mm.

Thus, the pre-ceramic body (P2 or P2z) may be in the form of a disc 150 mm or 100 mm in diameter and 40 mm thick. For example, it may also be a block with the dimensions 100 mm×100 mm×40 mm.

According to a particular embodiment, the crystalline metal oxide particles in step (a1) are ZrO₂ particles having a mean size of between 3 nm and 25 nm; the dispersion in step (a1) comprises 0.4% to 1.5% by weight of binder, the pressure in step (b1) is between 5 bar and 50 bar; green body P1 in step (c1) is at least 5 mm thick.

According to another embodiment, in addition to the non-optional steps, the method PC2 (and optionally the method PC1 and/or PC3) may comprise steps:
(b1′) and (b3), or
(c1′) and (d1), or
(b1′), (b3) and (c1′), or
(b1′), (c1′) and (d1), or
(b1′), (b3) and (d1), or
(b1′), (b3), (c1′) and (d1), or
(b1′) and (d1), or
(b1′), or
(b3).

Ceramic Body P3 or P3z

Ceramic body P3 or P23z according to the invention is based on metal oxide, zirconium oxide for P3z. It is a crystalline (nanocrystalline) ceramic body, advantageously free of cracks.

The crystalline zirconium oxide ceramic body P3z has a mean grain size of less than 200 nm, a density of more than 99% and a mechanical strength of at least 600 MPa.

The term “mechanical strength” is advantageously understood to mean the mechanical biaxial bending strength that may be measured according to standard ISO 6872:2015 (“piston-on-three-balls strength tests” method), regardless of the body concerned (P1, P2 or P3).

As already indicated for P1 and P1z, the metal oxide may be doped.

The zirconium oxide is advantageously doped with 1.0 mol % to 15 mol % of yttrium oxide or with 5.0 mol % to 15 mol % of cerium oxide. In particular, it may be ZrO₂ doped with 2.5 mol % to 10.5 mol % of yttrium oxide. It may advantageously be doped with 1.0 mol % to 15 mol % of a mixture of both oxides.

During pre-sintering (e1) and/or sintering (f1), the mean pore size and/or mean grain size of the body may increase.

The ceramic body P3 or P3z has a mean pore size (advantageously for P3) of less than or equal to 20 nm, more advantageously of less than 15 nm. According to a particular embodiment, the mean pore size is advantageously between 1 nm and 20 nm, e.g., between 2 nm and 20 nm.

Unlike bodies P1, P1z, P2 and P2z, the pore size of body P3 or P3z may be measured by image analysis from scanning electron microscopy (SEM) images obtained by observing the surface of a section of body P3 or P3z (the surface of P3 or P3z is advantageously prepared by mirror polishing, followed by vibratory polishing).

Imaging may be performed by means of a SEM (Scanning Electron Microscope) microscope, e.g., of the field emission type, in particular, at a voltage of less than 5 kV.

The term “pore size” means the largest dimension of each pore that can be measured in the image, and the term “mean size” means the mean of at least 100 pore size values.

Furthermore, the ceramic body P3z has a mean grain size of less than 200 nm, more advantageously of between 50 nm and 200 nm and even more advantageously of between 70 nm and 160 nm.

The mean grain size can be measured by the linear interception method, in particular, with a correction factor of 1.56, according to ASTM E112 and EN 623-3 standards.

This measurement may be performed by image analysis from the SEM images already used for measuring the size of the pores.

Ceramic material P3z has a mean mechanical strength advantageously of between 600 MPa and 3000 MPa.

The material P3z has an opalescence advantageously of between 9 and 23, advantageously when the zirconia oxide is doped with 1 mol % to 12 mol % of yttrium oxide.

The material P3z has an opalescence advantageously of between 16 and 22, advantageously when the zirconia oxide is doped with 3.5 mol % to 6.5 mol % of yttrium oxide.

The material P3z advantageously has a transmittance of at least 47%, more advantageously of between 49% and 72%, at 780 nm, for a thickness of 1 mm, when the zirconia oxide is doped with at least 2.5 mol % of yttria.

The material P3z has a direct transmittance value (RHT) advantageously of at least 22%, more advantageously of between 22% and 53%, at 780 nm, for a thickness of 1 mm, when the zirconia oxide is doped with at least 2.5 mol % of yttria.

The material P3z has a Vickers hardness advantageously of more than or equal to 12 GPa.

Transmittance denotes the total forward transmittance (TFT), that is to say the sum of direct transmittances (corresponding to the real in-line transmittance (RIT)) and indirect transmittances (corresponding to the diffuse transmittance). It is advantageously measured at ambient temperature by means of a spectrophotometer, e.g., the Jasco Y-670 with a sample holder provided with an integration sphere. The direct transmittance is measured with a second sample holder without any integrating sphere.

The colour of body P3 or P3z is measured, advantageously according to standard ISO 28642:2016, in transmission mode and reflection mode by using a spectrophotometer (e.g., the Jasco Y-670 model), according to the CIELAB colour space introduced by the International Commission on Illumination (CIE), with a light source (illuminant) D65 (described in the standard ISO 11644-2:2007) in the visible range and a reference CIE observer 10° (described in the standard ISO 11644-1:2007). This colour space is composed of the coordinates L*, a*, b*. The L* value (0 to 100) is a measure of colour clarity, the a* value is a measure of the tendency towards a red (a* positive) or green (a* negative) colour, and the b* value is a measure of the tendency towards a yellow (b* positive) or green (b* negative) colour.

In reflection mode, the colour is measured by placing a white background in reference, behind body P3 or P3z.

Opalescence corresponds to the “parameter opalescence”-OP, obtained by the difference in colour of the sample measured according to the CIELAB colour space in transmission and reflection mode, using the following formula:

OP=[(a*_{T}−a*_{R})²+(b*_{T}−b*_{R})²]^{1/2}
the indices T and R respectively refer to the transmission and reflection modes.

The presence of at least one doping element, in particular, yttria, makes it possible to control the mechanical strength of the material P3z and its optical properties. The optical properties are also affected by the colour of the material that can be more or less yellowish (b* value more or less high) depending on the content of the dyes. For example, material P3z may have the characteristics mentioned in Tables 1 to 5 depending on the quantity and nature of the dopant.

TABLE 1
Mean mechanical strength (MPa)

| Material | Dopant (mol %) | Mean mechanical strength (MPa) |
|---|---|---|
| ZrO2 | Y₂O₃ (1.5 to 3.5) | >1500, advantageously 1700 to 2500 |
| ZrO₂ | Y₂O₃ (1.5 to 2.5) | >2000 MPa, advantageously 2100 to 2700 |
| ZrO2 | Y₂O₃ (1.0 to 2.5) | >2000 MPa, advantageously 2100 to 2700 |
| ZrO₂ | Y₂O₃ (3.5 to 4.5) | >800 MPa, advantageously 800 to 1150 |
| ZrO2 | Y₂O₃ (4.5 to 6.5) | >700 MPa, advantageously 750 to 850 |
| ZrO2 | Y₂O₃ (6.5 to 10) | >600 MPa, advantageously 600 to 750 |
| ZrO₂ | CeO₂ (8 to 9.5) | 3500 MPa, advantageously 550 to 1000 |
| ZrO2 | CeO₂ (9.5 to 10.5) | 2600 MPa, advantageously 650 to 1000 |
| ZrO2 | CeO₂ (10.5 to 15) | >800 MPa, advantageously 850 to 1100 |

TABLE 2
Opalescence

| Material | Dopant (mol %) | Opalescence |
|---|---|---|
| ZrO₂ | Y₂O₃ (1.5 to 6.5) | ≥9, advantageously 14 to 22 |
| ZrO₂ | Y₂O₃ (2.5 to 6.5) | >12, advantageously 14 to 22 |
| ZrO₂ | Y₂O₃ (3.5 to 6.5) | >15, advantageously 16 to 22 |

TABLE 3
Transmittance (absolute value of b*)

| Material | Dopant (mol %) | Transmittance |
|---|---|---|
| ZrO₂ | Y₂O₃ (2.5 to 12) | >47%, advantageously 53 to 72. to 780 nm |
| | | (1 mm. b* <2)_{—} |
| ZrO₂ | Y₂O₃ (2.5 to 12) | >47%, advantageously 49 to 72. to 780 nm |
| | | (1 mm. b* <9.5) |
| ZrO₂ | Y₂O₃ (3.5 to 12) | >49%, advantageously 49 to 72. to 780 nm |
| | | (1 mm. b* <9.5) |
| ZrO₂ | Y₂O₃ (3.5 to 12) | advantageously 51 to 72. to 780 nm |
| | | (1 mm. b* <9.5) |
| ZrO2 | Y₂O₃ (5.5 to 12) | >59%, advantageously 59 to 72. to 780 nm |
| | | (1 mm. b* <7) |
| ZrO2 | Y₂O₃ (7.5 to 12) | >66%, advantageously 66 to 72. to 780 nm |
| | | (1 mm. b* <7) |

TABLE 4
Direct transmittance (absolute value of b*)

| Material | Dopant (mol %) | RIT |
|---|---|---|
| ZrO₂ | Y₂O₃ (2.5 to 10) | >22%, advantageously 22 to 53, at 780 nm |
| | | (1 mm, b* <2) |
| ZrO₂ | Y₂O₃ (2.5 to 10) | >22%, advantageously 22 to 52, at 780 nm |
| | | (1 mm, b* <9.5) |
| ZrO₂ | Y₂O₃ (4.5 to 10) | >22%, advantageously 22 to 52, at 780 nm |
| | | (1 mm, b* <9.5 L |
| ZrO₂ | Y₂O₃ (5.5 to 10) | >35%, advantageously 35 to 52, at 780 nm |
| | | (1 mm, b* <7) |
| ZrO₂ | Y₂O₃ (7.5 to 10) | >48%, advantageously 48 to 52. to 780 nm |
| | | (1 mm, b* <7) |

TABLE 5
Vickers hardness

| Material | Dopant (mol %) | Vickers hardness |
|---|---|---|
| ZrO₂ | Y₂O₃ (1.0 to 10) | 12 to 14.5, advantageously >12 GPa |
| ZrO₂ | Y₂O₃ (1.8 to 10) | ⅛ à14.5, advantageously >12.8 GPa |
| ZrO₂ | Y₂O₃ (3.5 to 10) | 14 to 14.5, advantageously >14 GPa |

According to a particular embodiment, the ceramic body is based on zirconium oxide doped with 2.5 mol % to 3.5 mol % of yttrium oxide. In this case, it has a mechanical biaxial bending strength advantageously of at least 1500 MPa.

According to a particular embodiment, the ceramic body has a transmittance of more than 50% and a direct transmittance of more than 20%, when these properties are measured at a wavelength of 780 nm for a thickness of 1 mm.

According to a particular embodiment, the ceramic body is based on zirconium oxide doped with 3.5 mol % to 6.5 mol % of yttrium oxide, advantageously 3.5 mol % to 4.5 mol %. In this case, it has a mechanical biaxial bending strength advantageously of at least 800 MPa.

According to a particular embodiment, the ceramic body is based on zirconium oxide doped with 5.5 mol % to 10 mol %, advantageously 5.5 mol % to 6.5 mol %, of yttria, has a value b* (absolute value) of less than 7, e.g., between 4 and less than 7, advantageously of approximately 4, and has a transmittance of more than or equal to 62% and a direct transmittance of more than or equal to 35%, when these properties are measured at a wavelength of 780 nm for a thickness of 1 mm.

According to a particular embodiment, the ceramic body is based on zirconium oxide doped with 7.5 mol % to 10 mol % of yttrium oxide, has a value b* (absolute value) of less than 7, advantageously of between 4 and less than 7, and has a transmittance of more than or equal to 66.8% and a direct transmittance of more than or equal to 48.1%, when these properties are measured at a wavelength of 780 nm for a thickness of 1 mm.

According to a particular embodiment, the ceramic body is based on zirconium oxide doped with 10.5 mol % to 15 mol % of cerium oxide and has a mechanical biaxial bending strength of at least 800 MPa and a transmittance of more than 30% at a wavelength of 780 nm for a thickness of 1 mm.

Advantageously, ceramic body P3 or P3z is free of cracks. According to the invention, a material free of any number of cracks does not comprise any maximum number of cracks of more than 500 μm in size, more advantageously of more than 50 μm, even more advantageously of more than 30 μm.

Method PC3 for preparing ceramic body P3 Preparing ceramic body P3 involves the preliminary formation of green body P1 according to steps (a1) to (c1) of method PC1 (optionally (a1) to (c1′)), and optionally of the pre-ceramic body P2 according to steps (d1) to (e1) of method PC2. Ceramic body P3z may be produced according to the method PC3 which may also be followed to prepare ceramic bodies P3 from metal oxide other than ZrO₂.

Thus, the method PC3 for preparing a ceramic body P3 comprises the following steps:
(e2) optionally, shaping the pre-ceramic body P2 formed according to the PC2 method or green body P1 formed according to the PC1 method;
(f1) forming a ceramic body P3 by sintering green body P1 as in one of steps (c1), (c1′) or (d1) or by sintering the pre-ceramic body P2 as of step (e1) or (e2), by sintering as in step (f1) being performed at a temperature of between 900° C. and 1300° C., advantageously of between 1050° C. and 1250° C. more advantageously of between 1100° C. and 1200° C.

Thus, step (f1) may combine the debinding-pre-sintering-sintering steps.

Step (e2)

When the shape of the pre-ceramic body P2 does not correspond to the shape desired for the finished object, the pre-ceramic body P2 can be shaped prior to sintering step (f1).

The shaping may also be performed on green body P1, if necessary (step c′). Body P1 has mechanical properties lower than body P2, however they may be sufficient for the shaping step (e2) with suitable tools.

Pre-ceramic body P2 or green body P1 is advantageously shaped by a technique chosen from the group comprising computer-aided design and manufacture, CAD/CAM (computer-aided design and manufacture).

Step (f1)

Step (f1) of sintering the pre-ceramic body P2 makes it possible, in particular, to densify the material at a temperature and to limit (or even remove) the growth of grains.

When the initial amount of dispersant or binder is too large, sintering step (f1) is not sufficient and generates a partial densification and/or a mean pore size of more than 20 nm and generally of more than 50 nm in P3. The same phenomenon can be observed when at least one of steps for preparing the green body is not performed according to the invention (pressure filtration (b1), advantageously pressing (b1′) and/or (b3), demoulding (b2) and drying (c1)).

The duration of sintering (permanence at the sintering temperature, 900° C. to 1300° C., advantageously 1050° C. to 1250° C.) may be adjusted depending on the dimensions, in particular, the thickness, of body P2 or P2z. In particular, it may be between a few minutes and a few hours.

When the sintering temperature is between 1050° C. and 1250° C., sintering is advantageously performed for a period of between 30 minutes and a few hours, more advantageously of between 30 minutes and 280 minutes; and even more advantageously of between 60 minutes and 180 minutes.

The temperature rise and fall time can be adapted according to step (f1), and may be performed on body P1 or P2, and/or according to the temperature applied during step (e1).

When sintering is performed from body P2, the total duration of step (f1) is advantageously of between 200 minutes and 700 minutes, and more advantageously of between 200 minutes and 500 minutes. In which case, step (f1) has a temperature rise advantageously of between 0.05° C./min and 15° C./min, and more advantageously of between 1° C./min and 8° C./min. The gradient rise and gradient fall are independent of each other. The gradient fall may, in particular, be greater, e.g., around 3° C./min to 50° C./min.

When sintering is performed from body P1, step (f1) consists of performing combined debinding-sintering heat treatment. According to this embodiment, the debinding (d1) and pre-sintering (e1) steps are performed during sintering heat treatment (f1). In this case, green body P1 is directly ceramised, without isolating the pre-ceramic intermediate P2, and the temperature rise during step (f1) in the temperature range 30° C. to 550° C. is advantageously of between 0.05° C./min and 1° C./min, more advantageously of between 0.1° C./min and 0.5° C./min. The reaction temperature may, in particular, be of between 0.05° C./min to 15° C./min, more advantageously of between 1° C./min to 8° C./min. The gradient rise and gradient fall are independent of each other. The gradient fall may, in particular, be greater, e.g., around 3° C./min to 50° C./min.

Advantageously, sintering in step (f1) is performed at atmospheric pressure. It is preferably performed in the absence of an electric discharge or an electric arc.

In addition to the filtration pressure, the relative humidity (demoulding, drying) and the temperature (pre-sintering, sintering), the PC1, PC2 and PC3 methods do not require special conditions in terms of environment. Thus, these methods may be performed in an inert medium (e.g., under argon or under nitrogen) as in a reducing or oxidising medium, e.g., in air. In general, when the PC3 medium is inert or reducing, it is preferable to perform an oxidation step in an oxidising medium so as to avoid altering the colour of body P3.

The present invention also relates to the use of the ceramic material derived from the PC3 method, in particular, in the field of dentistry (crowns, bridges, implants, etc.) or in optical applications requiring a ceramic having a high transmittance and/or a high mechanical strength and/or a high refractive index.

The invention and the advantages resulting from it appear more clearly from the following figures and examples that are given to illustrate the invention and in non-limiting manner.

### DESCRIPTION OF THE FIGURES

FIG. 1 illustrates TEM images of the particles of the dispersions (a): D1, (b): D2 and (c): D3.

FIG. 2 illustrates the micro-structure observed, e.g., INV-1_P3.

FIG. 3 illustrates the micro-structure observed, e.g., INV-22_P3.

FIG. 4 illustrates the micro-structure observed, e.g., INV-11_P3.

FIG. 5 illustrates the micro-structure observed, e.g., INV-5_P3 and the size of one of the pores.

FIG. 6 illustrates the appearance of ceramic materials of zirconia according to the prior art and of ceramic zirconia according to the invention, observed by reflection (upper line) and by transmission (lower line) under the same lighting conditions and for a thickness of 1±0.05 mm: (a): zirconia 3 mol % yttria (TZ-3YSB-E-Tosoh), (b) zirconia 5.3 mol % yttria (Zpex Smile-Tosoh), (c) zirconia according to Example INV-1_P3, (d) zirconia according to Example INV-22_P3, (e) zirconia according to Example INV-11_P3.

FIG. 7 illustrates pre-ceramic body P2 according to Example INV-12_P1 (left) and according to Example INV-15_P1 (Tight)

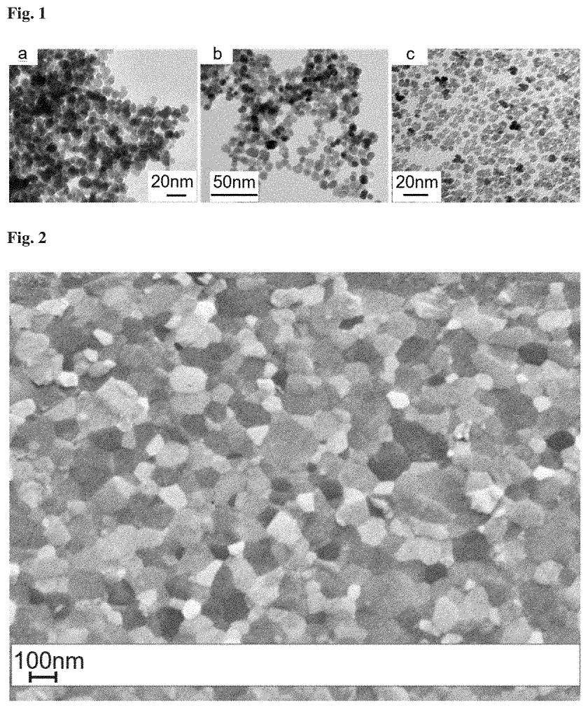

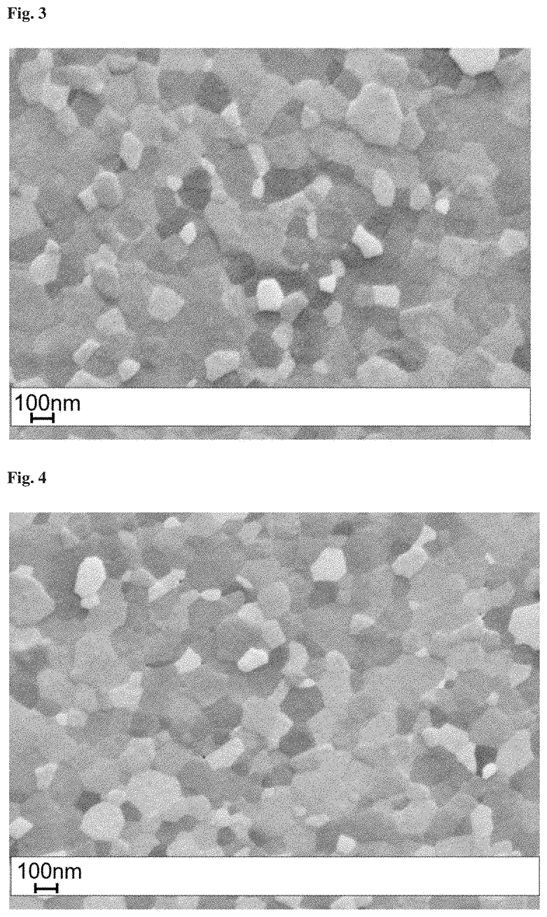

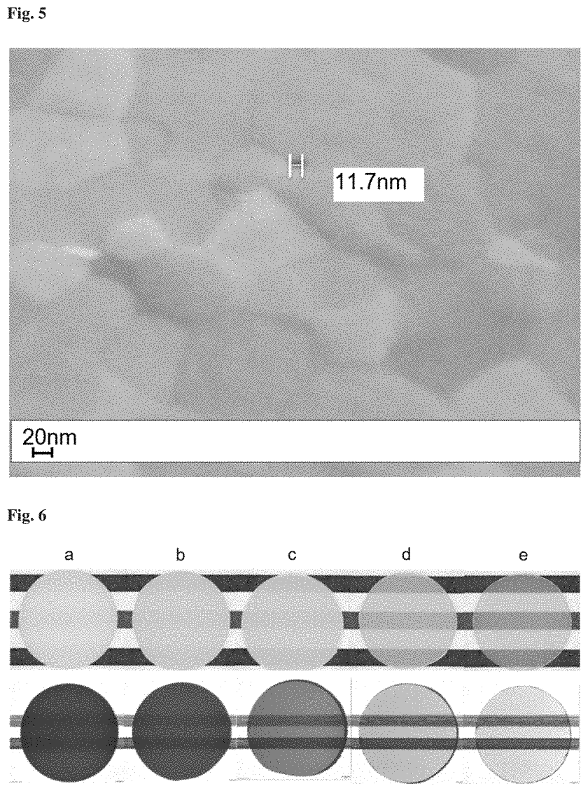

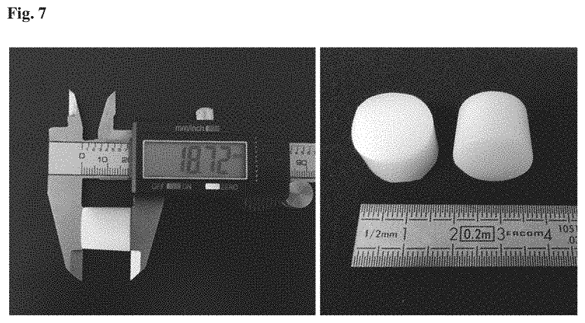

### EXAMPLES OF EMBODIMENTS OF THE INVENTION

A plurality of examples has been produced to illustrate methods PC1, PC2 and PC3, and also to illustrate bodies P1, P1z, P2, P2z, P3 and P3z.

The steps implemented to prepare these bodies are as follows:
(a1) providing a dispersion of crystalline metal oxide particles;
(b1) introducing the dispersion into a mould and forming a wet body by pressure filtration of the dispersion in the mould;
(b1′) optionally, pressing the wet body by pressure filtration;
(b2) demoulding the wet body;
(b3) optionally, removing the part of the wet body at the end of filtration, and optionally, the part at the beginning of filtration;
(c1) forming a green body by drying the wet body;
(c1′) optionally, shaping the green body;
(d1) optionally, debinding the green body;
(e1) optionally, forming a pre-ceramic body by pre-sintering the green body;
(e2) optionally, shaping the pre-ceramic body;
(f1) forming a ceramic body by sintering the pre-ceramic body.

According to certain examples, steps (d1), (e1) and (f1) are performed simultaneously.

In such a case, the green body is sintered directly, the debinding (d1) and pre-sintering (e1) steps being performed during sintering heat treatment (f1). In this case, the green body and the pre-ceramic body are therefore not isolated.

1/Dispersions Used

Typically, step (a1) consists of preparing a dispersion according to the following embodiment corresponding to dispersion D1 in Table 6:

A dispersion is prepared by sonication from 80 g of particles of ZrO₂ doped with 3.35 mol % Y₂O₃ and 8 nm in mean size (by number) in dispersion in water with the presence of a dispersant (40% by weight of particles+dispersant).

The metal oxide particles are advantageously prepared by hydrothermal treatment according to the protocol described in U.S. Pat. No. 8,337,788 or patent application FR 1872183. Where appropriate, the protocol is modified to incorporate the Y₂O₃, by means of the addition of a Y₂O₃ precursor, e.g., YCl₃, before the hydrothermal treatment. Once the particles are prepared by hydrothermal treatment, a dispersant (triammonium citrate, TAC) is added through stirring and sonication. The pH is adjusted by adding ammonia, then the dispersion is purified by dilution and concentration cycles. The concentration is performed by centrifugation when the dispersion is not stable and by tangential filtration when it has good colloidal stability. After purification, the dispersion is stable overtime. The dispersion is then concentrated to 40% by weight by tangential filtration.

This protocol is adjusted according to the data in Table 6 for preparing dispersions D2 to D22. Where appropriate, the synthesis protocol is modified to incorporate a dopant, by adding a Y₂O₃ or CeO₂ precursor before the hydrothermal treatment.

However, in dispersion D6, the dispersion resulting from the hydrothermal treatment is concentrated by centrifugation. The supernatant is removed and the functionalisation is performed by diluting the pellet with isopropanol in the presence of MEEEA. The dispersion is then purified by dilution by tangential filtration and concentrated by the same technique. In dispersion D5, the pH is adjusted by adding nitric acid.

TABLE 6
Characteristics of the dispersion in step (a1)

| Dispersion | Particles | Dispersant | Solvent | pH |
|---|---|---|---|---|
| D1 | YSZ-3.35 - 8 nm | TAC (3.7) | Water | 8.5 |
| D2 | YSZ-3.35 - 11 nm | TAC (3.5) | Water | 8.5 |
| D3 | YSZ-3.20 - 5 nm | TAC (6.2) | Water | 8.5 |
| D4 | YSZ-3.50 - 20 nm | TAC (5.5) | Water | 8.0 |
| D5 | YSZ-3.35 - 8 nm | MEEEA (3.5) | Water | 3 |
| D6 | YSZ-3.35 - 8 nm | MEEEA (8) | Isopropanol | — |
| D7 | YSZ-1.63 - 11 nm | TAC (3.8) | Water | 8.5 |
| D8 | YSZ-2.20 - 10 nm | TAC (3.9) | Water | 8.5 |
| D9 | YSZ-10.1 - 11 nm | TAC (4.2) | Water | 8.5 |
| D10 | YSZ-3.73 - 8 nm | TAC (3.7) | Water | 8.5 |
| D11 | YSZ-4.10 - 11 nm | TAC (3.7) | Water | 8.5 |
| D12 | YSZ-5.11 - 11 nm | TAC (3.6) | Water | 8.5 |
| D13 | YSZ-6.12 - 11 nm | TAC (4.1) | Water | 8.5 |
| D14 | YSZ-8.13 - 11 nm | TAC (4.1) | Water | 8.5 |
| D15 | YSZ-mix1 | TAC (4.4) | Water | 8.5 |
| D16 | CeZ-8.9 - 8 nm | TAC (5.4) | Water | 9 |
| D17 | CeZ-10 - 8 nm | TAC (5.1) | Water | 9 |
| D18 | CeZ-11.1 - 8 nm | TAC (5.1) | Water | 9 |
| D19 | CeZ-12.2 - 8 nm | TAC (5.4) | Water | 9 |
| D20 | CeZ-13.31 - 8 nm | TAC (5.4) | Water | 9 |
| D21 | CeZ-mix1 | TAC (—) | Water | 8.5 |
| D22 | CeZ-mix2 | TAC (—) | Water | 8.5 |

YSZ-3.35 - 8 nm: particles of ZrO₂ doped with 3.35 mol % Y₂O₃ and having a size of 8 nm
YSZ-mix1: mixture, having a ratio of 80/20 by weight, of particles of ZrO₂ doped with 6.12 mol % Y₂O₃ of 8 nm and particles of ZrO₂ doped with 2.20 mol % Y₂O₃ of 11 nm CeZ-mix1: mixture, having a ratio of 20/80 by weight, of particles of ZrO₂ doped with 13.31 mol % CeO₂ of 8 nm (dispersion D20) and particles of ZrO₂ doped with 5.11 mol % Y₂O₃ of 11 nm (dispersion D12)
CeZ-mix2: mixture, having a ratio of 20/80 by weight, of particles of ZrO₂ doped with 13.31 mol % CeO₂ of 8 nm (dispersion D20) and particles of ZrO₂ doped with 3.35 mol % Y₂O₃ of 11 nm (dispersion D2)
TAC (3.7): triammonium citrate, used at 3.7% by weight relative to the weight of the particles
TAC (—): % by weight of triammonium citrate = weighted mean of the constituents of the CeZ-mix1 and CeZ-mix2 mixtures
MEEEA: [2-(2-methoxyethoxy)ethoxy] acetic acid

2/Preparing Green Bodies by Pressure Filtration by Means of a Fluid

2.1/a Plurality of Green Bodies were Prepared by Modifying Steps (a1) to (c1) according to the parameters of Table 7.

In Example INV-1_P1, a solution of 0.72 g (0.9% by weight relative to the weight of the particles) of binder in 27.85 g of deionised water is added to 200 g of the dispersion D1, drop by drop for 30 minutes through stirring. The dispersion, containing 35% by weight of particles, is kept under sonication for 2 hours.

This dispersion is then poured into 8 identical moulds in assembly. The assembly rests on a rigid porous support in a vertical filtration system, in which the filtration is performed from top to bottom and the filtered solvent is discharged downwards through the porous support. Each mould used during step (b1) (and optionally (b1′)) is cylindrical in shape (20 mm in diameter). A 100 μm thick polycarbonate filter with a pore size of 100 mm to 200 nm (Nucleopore™ Whatman) is placed between the porous support and the dispersion. The system is closed so as to be hermetically sealed and connected to a pressurised gas circuit (argon, air or nitrogen). The pressure is gradually increased (10 minutes) until the value indicated in Table 7 is reached, and the dispersion is filtered under pressure. The pressure filtration is therefore performed by means of a fluid, in this case a gas. However, identical results may be obtained by means of a piston when the dispersion is excessive.

The pressure is gradually released (10 minutes). The still-closed filtration system is transferred to an environment having a relative humidity of more than 80%, the excess dispersion that has not been filtered is recovered and each wet body is demoulded by means of a polytetrafluoroethylene (PTFE) cylinder by simple pressure. The bodies prepared are in the form of a block, have the consistency of a rigid wet body in their lower part (part at the beginning of filtration), and the consistency of a soft gel in their part at the end of filtration.

During step (b3), the part at the end of filtration is removed by means of a spatula over a thickness of approximately 2 mm. The part at the beginning of filtration is removed over a thickness of 1 mm. The body is deposited on a support (PTFE grid) and inserted into a climatic chamber already at a relative humidity of 95% and at 30° C. The relative humidity is maintained at 95±3% during the first 24 hours, then gradually decreased with a gradient of 0.41%/h during days 2, 3, 4 and at 1%/h during day 5, to reach the final value of 40%. The green body obtained, in the form of a block, is free of cracks, has a translucent and opalescent appearance (orange if observed by transmission, bluish if observed by reflection) and has a visible deformation relative to the initial shape. This deformation is characterised by the presence of a curvature at the upper and lower surfaces, the upper surface being slightly concave and the lower surface slightly convex.

This protocol is adjusted to prepare green bodies according to the data in Table 7 using the dispersions presented in Table 6. For each example, an amount of excess dispersion is introduced into the mould and filtration is performed for 15 minutes to 24 hours depending on the thickness intended, so as not to filter the entire dispersion. When the drying time is longer, the relative humidity reduction gradients are adjusted proportionally to the time.

TABLE 7
Green bodies prepared from metal oxide particle dispersions by fluid pressure filtration

| | | | (b1) | (b2) | | (c1) | P1 | | |
|---|---|---|---|---|---|---|---|---|---|
| | | (a1) | Pressure | Wet | | Drying | Density | | Thickness |
| Disp. | P1 | Binder | (bar) | body | (b3) | (days) | (%) | Cracks | (mm) |
| D1^{(a)} | INV-1_P1 | 0.9 | 20 | CH + GM | Yes | 5 | 51 | No | 6 |
| D1^{(a)} | INV-2_P1 | 0.9 | 5 | CH + GM | Yes | 5 | 47 | No | ≥3 mm |
| D1^{(a)} | INV-3_P1 | 0.9 | 10 | CH + GM | Yes | 5 | — | No | 5 |
| D2^{(a)} | INV-4_P1 | 0.9 | 20 | CH + GM | Yes | 6 | 54 | No | 6 |
| D2^{(b)} | INV-5_P1 | 0.9 | 20 | CH + GM | No | 5 | 53 | No | 2.5 |
| D2^{(a)} | INV-6_P1 | 0.9 | 45 | CH + GM | Yes | 5 | — | No | — |
| D3^{(c)} | INV-7_P1 | 0.9 | 20 | CH + GM | Yes | 6 | 60 | No | 5 |
| D4^{(a)} | INV-8_P1 | 0.9 | 20 | CH + GM | Yes | 5 | — | No | 5 |
| D6^{(d)} | INV-9_P1 | 0 | 20 | CH + GM | No | 20 | — | No | 2.5 |
| D7^{(a)} | INV-10_P1 | 0.9 | 20 | CH + GM | Yes | 5 | — | No | 5 |
| D14^{(a)} | INV-11_P1 | 0.9 | 20 | CH + GM | Yes | 5 | — | No | 5 |
| D1^{(a)} | CE1 | 0.4 | 20 | CH + GM | Yes | 6 | 65 | Yes | 6 |
| D1^{(a)} | CE2 | 0.9 | 80 | CH + GM | Yes | 6 | — | Yes | 6 |
| D1^{(a)} | CE3 | 0 | 80 | CH + GM | No | 6 | 67 | Yes | 6 |
| D1^{(a)} | CE4 | 0 | 40 | CH + GM | Yes | 6 | 66 | Yes | 6 |
| D2^{(a)} | CE5 | 0 | 3 | No body | NA | NA | NA | NA | NA |
| | | | | formed | | | | | |
| D2^{(a)} | CE6 | 0 | 20 | CH + GM | Yes | 4 | 53 | Yes | 6 |
| D2^{(a)} | CE7 | 0 | 20 | CH + GM | No | 6 | 54 | Yes | 6 |
| D2^{(a)} | CE8 | 0 | 20 | CH + GM | Yes | 6 | — | Yes | 8 |

CH + GM: wet body + soft gel
Dispersions at ^{(a)}35% or ^{(b)}40% or ^{(c)}30% or ^{(d)}51% by weight of particles relative to the total weight of the dispersion.
PVA (polyvinyl alcohol) in % by weight relative to the weight of the particles
Density: expressed in % relative to the theoretical density calculated according to the composition
NA: not applicable

In general, when demoulding (b2) is performed at less than 80% relative humidity, the green body has cracks of more than 500 μm.

When demoulding (b2) is performed at more than 80% relative humidity, and when drying (c1) is performed at less than 90%, the green body has cracks of more than 500 μm.

The examples show that a green body free of cracks may only be obtained for a combination of parameters, in particular, a sufficient quantity of binder and a specific filtration pressure range. Under optimum conditions, step (b3) may prove necessary to minimise the density gradient and avoid cracking when the green body is at least 3 mm to 4 mm thick (INV-4_P1 and INV-5_P1).

According to CE1, CE4 and CE6 to CE8, the presence of 0% or 0.4% by weight of binder is not sufficient to form a green body free of cracks, but in the case of a thickness of at least 5 mm.

According to CE2 and CE3, the filtration pressure is too high (80 bar) to avoid cracks being formed, in the presence or not of binder, for a green body at least 5 mm thick.

In CE5, there is no formation of a solid wet body because the pressure is not high enough to form a wet body.

Green body INV-7_P1 has a mean pore size (BJH) of 3.4 nm and a specific surface area (BET) of 140 m²/g.

Green body INV-4_P1 has a mean pore size (BJH) of 4.9 nm and a specific surface area (BET) of 114 m²/g.

Green body INV-1_P1 has a mean pore size (BJH) of 4.2 nm and a specific surface area (BET) of 117 m²/g.

2.2/Binders distinct from the PVA were also used to form the green body. These examples are given in Table 8.

TABLE 8
Green bodies prepared from dispersions of metal oxide
particles by pressure filtration by means of a piston

| | | | | (b2) | | (c1) | P1 | |
|---|---|---|---|---|---|---|---|---|
| | | (a1) | (b1) | Wet | | Drying | | Thickness |
| Disp. | P1 | Binder^{(d)} | Pressure | body | (b3) | (days) | Cracks | (mm) |
| D2^{(b)} | INV-21_P1 | 1 PVP | 20 | CH + GM | Yes | 5 | No | 4 |
| D5^{(c)} | INV-22_P1 | — | 40 | CH + GM | No | 5 | No | 3 |
| D1^{(a)} | INV-23_P1 | 0.4PVP + | 20 | CH + GM | Yes | 5 | No | 5 |
| | | 0.4 PEG | | | | | | |
| D1^{(a)} | INV-24_P1 | 0.75 PVA + | 20 | CH + GM | Yes | 5 | No | 5 |
| | | 0.8 glycerol | | | | | | |

Dispersions at ^{(a)}30% or ^{(b)}35% or ^{(c)}44% by weight of particles ^{(d)}in % by weight relative to the weight of the dispersion
CH + GM: wet body + soft gel
PVP: polyvinyl pyrrolidone
PEG: polyethylene glycol

3/Preparing Green Bodies by Centrifugation

56 ml of the D2 dispersion were separated into 4 equal parts, and acetic acid was added to modify the pH. The amount of acetic acid varies from one sample to another to obtain dispersions having a pH of between 5.5 and 8.5. The pH of the 4 dispersions after adding was 8.5, 7.5, 6.5 and 5.5. The 4 dispersions were centrifuged, at 30,000 g for 10 minutes, in cylindrical centrifugation pots having a volume of 50 ml, so as to separate the particles that form a solid precipitate, and the dispersion solvent (supernatant). The supernatant is then removed and the precipitate is dried by a drying cycle similar to that presented in Example INV-4_P1. The tests result in green bodies having numerous cracks. These green bodies are not suitable for forming a pre-ceramic or ceramic material having a thickness of several mm.

The cracked green bodies (thickness <5 mm) were recovered and subjected to debinding/sintering treatment as in Example INV-3_P3. The cracked pieces obtained had a translucent appearance and a density of between 99% and 99.8% relative to the theoretical density.

This method makes it possible to obtain good densification of the cracked bodies after sintering, but does not make it possible to obtain green body P1 or P1z that is free of cracks.

4/Preparing Green Bodies by Gel Casting

100 ml of the dispersion D2 were prepared, but without binder. The dispersion was separated into 5 equal parts and heated to 80° C. through stirring in a closed container. Incremental amounts of gelling agent (purified agar) of between 0.2% and 1.2% by weight relative to the mass of the particles were introduced progressively into the dispersion through stirring. The dispersion was then degassed under vacuum and poured into silicone moulds 30 mm in diameter and cooled to ambient temperature, forming a solid gel. The gel was then dried according to a drying cycle similar to that presented in Example INV-4_P1. After drying, the green bodies were observed. In the case of low gelling agent contents (less than 0.8% by weight), the gel did not retain the cylindrical shape of the mould. In the case of higher gelling agent contents (between 0.8% and 1.2% by weight), a green body of cylindrical shape without apparent cracks was obtained. All the green bodies were subjected to debinding/sintering treatment as in Example INV-3_P3. The green bodies free of cracks made it possible to obtain a sintered body with a density of 92% to 96% of the theoretical density and a partially translucent appearance. Analysis of a polished section of these sintered bodies has made it possible to show the presence of numerous residual pores with a size of between 20 nm and 100 nm as well as numerous macropores with a size of between 5 μm and 100 μm. The cracked green bodies made it possible to obtain small-sized sintered bodies and with a density of between 96.0% and 97.6% of the theoretical density, with a reduced presence of both pore families (20 nm to 100 nm and 5 μm to 100 μm).

These tests result in green bodies having a fairly high density and numerous cracks, or green bodies having a low density and macroporous defects.

These green bodies are not suitable for forming a pre-ceramic or ceramic material having a thickness of several mm.

5/Preparing Bodies by Extrusion and Micro-Extrusion

30 ml of the dispersion D2 were prepared, but without binder. The dispersion was then reconcentrated by osmotic compression according to the following steps:

800 ml of a 20% by weight solution of PEG8000 (M=8000 g/mol) in water were prepared and then adjusted to pH 8.5 by adding a 30% by weight aqueous ammonia solution.

The dispersion was transferred into a dialysis membrane in the form of a tube closed at the ends (Spectra/Por supplied by Spectnumlab) with a nominal cut-off threshold of molecular weight 12-14 kD. This dialysis membrane was then placed in the PEG8000 solution previously prepared. The dispersion was thus dialysed against this PEG8000 solution to extract a portion of the water present in the zirconia dispersion and thus concentrate it.

After approximately 9 hours of dialysis, a colloidal paste of nanoparticles of zirconia at 66% by weight in water was recovered. The colloidal paste was then homogenised and deaerated using a planetary mixer with asymmetric double axes under vacuum (Speedmixer DAC150.1 FVZ-K, Hauschild Engineering). The rheological properties of this paste show a shear-thinning behaviour (viscosity decreasing with the shear rate). Measurements by oscillatory rotational rheometry in plane-plane geometry at a frequency of 1 Hz have shown that this paste behaves like a solid at rest, with an elastic (or conservation) modulus G′ of approximately 6×10⁵ Pa, and a viscous (or loss) modulus of approximately 3×10³ Pa, having a yield point of approximately 6500 Pa. These properties make it possible to shape the paste by extrusion or by additive manufacturing by means of microextrusion.

A 5 cm³ tubular cartridge was filled with this paste and then centrifuged using a planetary mixer with asymmetrical double axes to extract the air which may remain trapped between the paste and the wall of the cartridge. A cylinder (12 mm in diameter and 16 mm in height) was produced by microextrusion of the paste in filament form through a 250 μm diameter nozzle screwed onto the syringe. The microextrusion was controlled by means of a piston with a controlled speed of movement. The cartridge-piston assembly is mounted on a printing machine which may control its movement in the three spatial directions and perform microextrusion in an environment with a relative humidity of more than 95%. A 10 cm³ cartridge was filled and centrifuged in a similar manner, then the end part of the cartridge was cut and a cylinder (18 mm in diameter and 25 mm in height) was manufactured by extruding the contents of the cartridge onto the same support used for drying, in Example INV-4_P1. The two objects were dried according to a drying cycle similar to that in Example INV-4_P1. Both green bodies had no cracks and had a translucent appearance. Both green bodies were debonded and pre-sintered according to the protocol used in Example INV-2_P2, forming two pre-ceramic bodies. The body obtained by extrusion had a macroscopic crack, whereas the body obtained by microextrusion had no cracks. Unlike the pre-ceramic bodies in Examples INV-1_P2 to INV-11_P2, both bodies had lost their translucent appearance.

Discs 2 mm thick of both pre-ceramic bodies were subjected to the sintering conditions in Example INV-19_P3. After sintering, the discs in both cases had a translucent appearance in their outer part, over a thickness of approximately 3 mm, and an opaque appearance in their inner part. MEB observations after vibratory polishing revealed a microstructure having a mean grain size of 130 nm, a mean pore size of less than 20 nm in the translucent outer part, and a mean pore size of more than 50 nm in the opaque inner part.

This technique does not make it possible, starting from a dispersion of nanoparticles with a size of less than 40 nm, to obtain both green bodies free of cracks, a good densification of the material without the presence of a densification gradient and the absence of nanopores with a mean size of more than 20 nm in the sintered piece.

These green bodies are not suitable for forming a pre-ceramic or ceramic material having a thickness of several mm.

5B/Preparing Bodies by Vacuum Filtration

A plaster mould (20 mm in diameter and 10 mm in height) was filled with 20 ml of dispersion D2, and placed under vacuum between 1 mbar and 10 mbar. After 24 hours of operation, the mould was emptied of the still fluid dispersion. An extremely thin layer (<1 mm) of zirconia had been deposited on the surface of the plaster. The layer cracked into several parts during drying performed according to the conditions in Example INV-4_P1.

This technique does not make it possible to obtain a green body with a satisfactory thickness starting from a dispersion of nanoparticles with a size of less than 40 nm.

6/Preparing Green Bodies by Double Pressure Filtration

This section shows that pressing step (b1′) may prove to be essential to form a green body of at least 5 mm thick and free of cracks. Step (b3) may also be performed, but it is not necessary when pressing is performed by means of a piston.

6.1/Filtrations (b1) and (b′) by Means of a Dispersion

Green bodies prepared from dispersions of metal oxide particles by pressure filtration by means of a fluid (b1+b1′).

In Example INV-12_P1, filtration (b1) is performed by means of a first dispersion D2, at a concentration of 35% by weight, used to form the green body at 20 bar. When the desired wet body thickness is reached (72 hours), the pressure is returned to atmospheric pressure, the filtration device is opened and the remaining dispersion is removed and replaced by a second dispersion, D1, at a concentration of 25% by weight, to perform a new filtration cycle for 12 hours. During the second cycle, which corresponds to pressing step (b1′), the tiltration rate of dispersion D1 is lower than that of dispersion D2. Under these conditions, the body formed from the first dispersion D2 is compacted and the density gradient is reduced. The following steps are performed as in Example INV-1_P1. During demoulding, step (b3) is performed to remove the upper part of the wet body, of variable thickness according to the dispersion used and the pressing time, corresponding to the part formed with the second dispersion. After drying (6 days), green body P1 in the form of a cylinder with a thickness of 18.7 mm is free of cracks and has a very slight curvature at the upper and lower surfaces and a very slight inclination of the lateral surface.

In all the examples in Table 9, the pressing step (b1′) is performed for a period equal to 10% to 25% of the filtration period in step (b)

TABLE 9
Green bodies prepared from dispersions of metal oxide particles by
double pressure filtration by means of a dispersion of particles.

| | | | (b1) | | (b1′) | (c1) | | P1 | |
|---|---|---|---|---|---|---|---|---|---|
| | | (a1) | Pressure | (b1′) | Pressure | Drying | | | Thickness |
| Disp. | P1 | Binder | (bar) | Disp.2 | (bar) | (days) | (b3) | Cracks | (mm) |
| D2^{(a)} | INV-12_P1 | 0.9 | 20 | D1^{(b)} | 20 | 6 | Yes | No | 18.7 |
| D12^{(a)} | INV-13_P1 | 0.9 | 20 | D1^{(b)} | 20 | 6 | Yes | No | 15 |
| D1^{(a)} | INV-19_P1 | 2 | 20 | D1^{(b)} | 40 | 6 | Yes | No | 15 |
| D2^{(a)} | INV-20_P1 | 1.8 | 20 | D1^{(b)} | 20 | 6 | Yes | No | 22 |

Binder: PVA in % by weight relative to the weight of the dispersion
Dispersions at ^{(a)}35% or ^{(b)}25% or ^{(c)}30% by weight of particles

The green bodies in the examples in Table 9, in the form of a cylinder, are very slightly deformed after drying and are free of cracks.

6.2/Filtration (b1) and (b1′) by Means of a Piston

Green bodies were also prepared from dispersions of metal oxide particles by double pressure filtration by means of a piston. In this case, the pressure is applied by means of a PTFE piston, with a diameter equal to the diameter of the mould and provided with an O-ring to guarantee sealing, directly in contact with the dispersion. The force exerted by the piston is controlled to ensure a constant pressure during step (b1).

In Example INV-14_P1, 30 ml of dispersion D1 are prepared with the addition of 0.9% by weight of PVA binder as in Example INV-1_P1. The resulting dispersion is diluted to 30% by weight. 6 ml of the dispersion are poured into a cylindrical mould 20 mm in diameter, in a vertical filtration system similar to that in Example INV-1_P1. A PTFE piston is inserted into the upper part of the mould and brought into contact with the dispersion. A force corresponding to a pressure of 20 bar is then applied. The entire dispersion is filtered in step (b1), which lasts 3 hours. The force is maintained during pressing step (b1′) lasting 1 hour. The force was then withdrawn and the body is demoulded under the same conditions as, e.g., INV-1_P1, by exerting a slight pressure on the piston. The wet body obtained has the consistency of a rigid body over its entire thickness. Step (b3) is not performed. Drying is then performed as in Example INV-12_P1 for 6 days. The resulting green body, translucent and opalescent, is free of cracks. It does not have any curvature at the upper and lower surfaces, and retains the block shape obtained after step (b2).

The examples of Table 10 are performed by the same technique, by varying the initial dispersion and the durations of steps (b1) and (b1′) to obtain different thicknesses for body P1. In all the examples, the same binder is used, as well as the same drying protocol.

TABLE 10
Green bodies prepared from dispersions of metal oxide particles
by double pressure filtration by means of a piston.

| | | | | | | | (b2) | | | | |
|---|---|---|---|---|---|---|---|---|---|---|---|
| | | (b1) | | | | | Cracks | (c1) − P1 | | | |
| | | Pressure | (b1) | | (b1′) | | after | | | Thickness | Density |
| Disp. | P1 | (bar) | Duration | | Duration | | (b2) | Cracks | Gradient | (mm) | (%) |
| D1^{(c)} 6 ml | INV-14_P1 | 20 | 3 | h | 1 | h | No | No | No | 4 | 51.5 |
| D2^{(a)} 16 ml | INV-15_P1 | 20 | 48 | h | 15 | h | No | No | No | 12 | 49.3 |
| D2^{(a)} 29 ml | INV-16_P1 | 20 | 98 | h | 26 | h | No | No | No | 21 | 48.4 |
| D3^{(c)} 6 ml | INV-17_P1 | 20 | 3.5 | h | 1.5 | h | No | No | No | 4 | 55.4 |
| D18^{(d)} 29 mL | INV-18_P1 | 20 | 65 | h | 17 | h | No | No | No | 22 | 53.2 |

In Table 10, the dispersions comprise ^{(a)} 35% or ^{(b)} 25% or ^{(c)} 30% or ^{(d)} 38% by weight of particles. The term “Gradient” indicates the visual observation of a curvature of the lower and upper surfaces of the green body, associated with a difference in diameter. This curvature indicates the presence of a density gradient in the wet body. As already indicated, the curvature of the lower and/or upper surfaces indicates the presence of a deformation relative to the initial shape, namely obtaining a slightly concave upper surface and/or a slightly convex lower surface. The density is indicated in % relative to the theoretical density.

Bodies P1 in Example INV-15_P1 were analysed by XRD (diffractometer mod. Bruker D8 Advance) and the Scherrer method was applied to calculate the size of the crystallites. The size of the crystallites is 10.11 nm. The diffraction pattern has only peaks corresponding to the quadratic/cubic phase.

7/Preparing Pre-Ceramic Bodies

Pre-ceramic bodies were prepared by combined heat treatment of debinding-pre-sintering from green bodies according to the data of Table 11. When the green bodies had a curvature, they were flattened manually by means of a silicon carbide polishing disc of grain 640 in the absence of water or lubricant.

The green bodies were deposited in porous alumina containers and inserted into a conventional muffle-type furnace with ceramic heating bodies (mod. Nabertherm L 9/11 BO). The heat treatment applied was as follows:
0.1° C./min from ambient temperature up to 200° C.
plateau at 200° C. for 3 hours 25-0.2° C./min from 200° C. to 400° C.
plateau at 400° C. for 3 hours
0.2° C./min at 400° C. at the pre-ceramic body formation temperature
plateau at the pre-ceramic body formation temperature for the indicated time
cooling to 0.5° C./min up to ambient temperature.

TABLE 11
Preparing pre-ceramic bodies

| | | | | | | P2 | | | | |
|---|---|---|---|---|---|---|---|---|---|---|
| | | | | | | | | | | Specific |
| | | | P2 | (e1) | | | | | Pore | surface |
| | | | Thickness | | Duration | Density | | Mech. | size | area |
| Disp. | P1 | P2 | (mm) | (° C.) | (h) | (%) | Hardness | res. | (BJH) | (BET) |
| D2 | INV-4_P1 | INV-1_P2 | 6 | 500 | 1 | 59.5 | — | — | 6.5 | 67 |
| D1 | INV-1_P1 | INV-2_P2 | 6 | 550 | 1 | 56.9 | 150 | 25 | — | — |
| D1 | INV-1_P1 | INV-3_P2 | 6 | 600 | 1 | 57.9 | 164 | 29 | 6.5 | 68 |
| D1 | INV-1_P1 | INV-4_P2 | 6 | 650 | 1 | 59.6 | 180 | 36 | — | — |
| D1 | INV-1_P1 | INV-5_P2 | 5.5 | 800 | 1 | 66.1 | — | — | 8.6 | 26.5 |
| D3 | INV-7_P1 | INV-6_P2 | 5 | 550 | 1 | 62.9 | — | — | — | — |
| D2 | INV-12_P1 | INV-7_P2 | 20 | 800 | 1 | 67.8 | — | — | — | — |
| D1 | INV-14_P1 | INV-8_P2 | 4 | 500 | 1 | 56.1 | — | — | 5.0 | 120 |
| D2 | INV-15_P1 | INV-9_P2 | 12 | 600 | 1 | 62.0 | — | — | 7.3 | 57 |
| D2 | INV-16_P1 | INV-10_P2 | 20 | 750 | 1 | 65.9 | 210 | 43 | — | — |
| D18 | INV-18_P1 | INV-11_P2 | — | 550 | 3 | 57.1 | — | — | — | — |

| | | | | | | |
|---|---|---|---|---|---|---|
| D1 | INV-19_P1 | CE9 | 15 | 550 | 1 | No P2 formation |

Mech. res.: mechanical biaxial bending strength in MPa
Hardness: Vickers HV1 hardness measured with a load of 1 kgf (in Vickers units)
Pore size: size of the interconnected porosity in nm (BJH method)
Specific surface area: in m²/g (BET method)
Density: in % relative to the theoretical density
CE9 shows that, when the body is more than 5 mm thick, it is preferable to limit the quantity of binder to avoid cracking.
Bodies P2 of Examples INV-2_P2 and INV-9_P2 were analysed by XRD (diffractometer mod. Bruker D8 Advance). The diffraction patterns have only peaks corresponding to the quadratic/cubic phase. The size of the crystallites measured by the Scherrer method is 10.11 nm for INV-2_P2 and 12.06 nm for INV-3_P2.

A section of body P2 in Example INV-2_P2 was polished by ionic polishing using an ionic polisher with Argon ion beams (mod. Ilion II—Gatan) and observed by SEM. The observations revealed that the size measured by XRD corresponds approximately to the size of the grains that comprise the microstructure of the pre-ceramic body.

8/Shaping Pre-Ceramic Bodies by CFAO

The pre-ceramic bodies were bonded on a support compatible with a dental CAD/CAM system of the Cerec (Denstply Sirona) type, represented by a multi-axis milling machine, and machining tests were performed with protocols typical of dental machining. One of these protocols is represented by milling, using a tool with a defined cutting geometry, commonly used in machining zirconia blocks, available on the market with a defined maximum forward speed. One second protocol is represented by “grinding”, using an abrasive tip tool, commonly used in machining glass-ceramic dental blocks, available on the market, with a defined maximum speed.

The examples in Table 12 summarise the results of machining on pre-ceramic bodies pre-sintered at different temperatures, according to one of the two protocols, with or without water cooling. In the case where there are no cracks or chipping on the body after step (c1), the pre-ceramic body is retained and declared compatible with the dental CAD/CAM.

TABLE 12
Compatibility of pre-ceramic bodies with shaping techniques

| | | (e1) | (c1′) | Cooling | CAD/ |
|---|---|---|---|---|---|
| | | Pre- | Protocol | with | CAM |
| P1 | P2 | sintering^{(d)} | % speed^{(c)} | water^{(a)} | accounting |
| INV-15_P1 | INV-12_P2 | 500 | G - 100% | Yes | Yes |
| INV-4_P1 | INV-13_P2 | 550 | G - 100% | Yes | Yes |
| INV-16_P1 | INV-14_P2 | 600 | G - 100% | Yes | Yes |
| INV-4_P2 | INV-15_P2 | 650 | G - 100% | Yes | Yes |
| INV-1_P1 | INV-16_P2 | 650 | G - 100% | Yes | Yes |
| INV-16_P1 | INV-17_P2 | 700 | G - 100% | Yes | Yes |
| INV-12_P1 | CE17 | 850 | G - 100% | Yes | No, |
| | | | | | chipping |
| INV-12_P1 | CE18 | 550 | F^{(b)} - 50% | Yes | No, |
| | | | | | chipping |
| INV-12_P1 | CE19 | 550 | G - 80% | No | No, |
| | | | | | chipping + |
| | | | | | cracks |

^{(a)}cooling during CAD/CAM shaping
^{(b)}milling
^{(c)}% of maximum speed according to protocol; G = grinding; F = milling
^{(d)}pre-sintering temperature in ° C.

In the specific case of CAD/CAM machining, Example CE17 shows that an excessive pre-sintering temperature (850° C.) leads to cracks being formed during machining. Examples EC18 and EC19 show that machining conditions, in particular, the speed or absence of cooling, may also generate cracks. The person skilled in the art will be able to adapt the machining conditions (speed and cooling) according to their general knowledge.

9/Preparing Ceramic Bodies

9.1/Ceramic bodies were prepared from green bodies, without isolating bodies from the intermediate debinding (d1) and pre-sintering (e1) steps. To minimise the machining steps on the sintered material, the thickness of the green bodies was reduced by manual machining to 2 mm by means of a silicon carbide polishing disc of grain 640, in the absence of water or lubricant.

The heat treatment is performed in a high-temperature furnace of the muffle type with heating elements made of MoSi₂ (mod. Nabertherm LHT 03/17 D). The treatment is performed in a manner identical to Example INV-4_P2 up to 650° C., then a gradient of 3° C./min is applied up to the sintering temperature, then the temperature is maintained for a plateau time. The sintered body is then cooled at a rate of 50° C./min to ambient temperature.

After sintering, the ceramic bodies were pre-polished with diamond pre-polishing discs (MD-Piano, Struers) of grain 120 to 1200, mounted on a polishing machine, to reduce their thickness. Then, they were polished with diamond dispersions by means of polishing discs until a mirror finish was obtained. The resulting discs, 1.2±0.2 mm thick, were characterised in terms of mechanical properties (hardness, mechanical strength) and microstructure (grain size measured by SEM after vibratory polishing). Discs with a thickness of 1.00±0.05 mm, prepared in a similar manner, were characterised in terms of optical properties (value b*, CR, OP, TP).

The results of the characterizations are reported in Tables 14v and 15. In the examples where body P1 is not indicated, body P1 is obtained in a manner identical to Example INV-4_P1, except that each dispersion is different.

In Examples INV-2_P3, INV-4_P3, INV-5_P3, bodies are sintered by the “two-step sintering” method, with a short plateau at a higher temperature followed by a longer plateau at a lower temperature.

TABLE 13
Ceramic bodies

| | | | | P3 | | | |
|---|---|---|---|---|---|---|---|
| | | | | | Vickers | | Grain |
| | | | (f1) | Density | Hardness | Mech. | size |
| Disp. | P1 | P3 | Sintering | (%) | (GPa) | res. | (nm) |
| D1 | INV-20_P1 | INV-1_P3 | 2 hours at 1150° C. | 99.9 | — | — | 117 |
| D1 | INV-20_P1 | INV-2_P3 | 2 minutes at 1200° C. + | 99.8 | 12.7 | | 103 |
| | | | 10 hours at 1000° C. | | | | |
| D2 | INV-21_P1 | INV-3_P3 | 2 hours at 1150° C. | 99.9 | — | — | 131 |
| D2 | INV-21_P1 | INV-4_P3 | 1100° C. 60 min + | — | 13.0 | 2620 | 120 |
| | | | 1000° C. 10 hours | | | | |
| D2 | INV-21_P1 | INV-5_P3 | 18 minutes at 1250° C. + | 99.9 | 14 | 2215 | 145 |
| | | | 10 hours at 1000° C. | | | | |
| D2 | INV-21P1 | INV-6_P3 | 2 hours at 1200° C. | 99.9 | 13.7 | 1900 | 134 |
| D4 | INV-8_P1 | INV-7_P3 | 2 hours at 1150° C. | 99.8 | — | — | 137 |
| D6 | INV-9_P1 | INV-8_P3 | 2 hours at 1150° C. | 99.9 | — | — | 124 |
| D7 | INV-10_P1 | INV-9_P3 | 2 hours at 1150° C. | — | 12.2 | 2550 | 151 |
| D12 | INV-13_P1 | INV-10_P3 | 1 hour at 1200° C. | 99.9 | 14.4 | 794 | 136 |
| D14 | INV-11_P1 | INV-11_P3 | 1 hours at 1200° C. | 99.9 | 14.3 | 680 | 133 |
| D16 | — | INV-12_P3 | 2 hours at 1150° C. | 99.8 | — | 595 | 113 |
| D17 | — | INV-13_P3 | 2 hours at 1150° C. | 99.7 | — | 780 | 111 |
| D18 | — | INV-14_P3 | 2 hours at 1150° C. | 99.7 | — | 910 | 115 |
| D19 | — | INV-15_P3 | 2 hours at 1150° C. | 99.9 | — | 952 | 126 |
| D20 | — | INV-16_P3 | 2 hours at 1150° C. | 99.8 | — | 923 | 115 |
| D21 | — | INV-17_P3 | 2 hours at 1175° C. | 99.8 | 13.9 | 740 | 129 |
| D22 | — | INV-18_P3 | 2 hours at 1150° C. | 99.9 | 13.6 | 840 | 122 |
| D10 | — | INV-19_P3 | 2 hours at 1150° C. | 99.8 | 13.9 | 1075 | 128 |
| D9 | — | INV-20_P3 | 3 hours at 1225° C. | 99.9 | — | — | 145 |
| D8 | — | INV-21_P3 | 2 hours at 1150° C. | — | 13.0 | 2360 | 129 |
| D13 | — | INV-22_P3 | 1 hour at 1200° C. | 99.9 | 14.2 | 731 | 136 |
| D15 | — | INV-23_P3 | 2 hours at 1175° C. | 99.9 | 14 | 675 | 134 |
| D11 | — | INV-24_P3 | 2 hours at 1175° C. | 99.8 | 14.1 | 890 | 113 |
| D2 | INV-20_P1 | CE21 | 2 hours at 1150° C. | 97.3 | — | — | — |

Sintering: sintering temperature and plateau time
Mech. res.: mean mechanical biaxial bending strength in MPa
Hardness: Vickers HV10 mean hardness measured in GPa
The mean pore size of bodies INV-1_P3 to INV-24_P3 is less than 15 nm.
EC21 (1.8% binder) shows that, for bodies more than 5 mm thick, it is preferable to limit the binder quantity. In fact, in the presence of an excessive quantity of binder, it is possible for the densification during sintering to be altered and for it to be impossible to obtain a nanocrystalline ceramic material with a density of more than 99%.

TABLE 14
Transmittance values of the ceramic materials

| | | | | | | | TFT 555 nm- | RIT 555 nm- |
|---|---|---|---|---|---|---|---|---|
| | | | | | | | 600 nm- | 600 nm- |
| Disp. | P1 | P3 | L*, a*, b* | CR | OP | TP | 780 nm | 780 nm |
| D1 | INV-23_P1 | INV-1_P3 | 94, −1.1, −0.8 | 0.47 | 17 | 26.1 | 43.4-45.8-56.0) | 6.9-11.9-35.2) |
| D1 | INV-23_P1 | INV-2_P3 | 90, −1.1, 9.1 | 0.62 | 20.3 | 20.7 | — | — |
| D2 | INV-21_P1 | INV-3_P3 | 94.1, −1.2, 0.4 | 0.48 | 14.6 | 25.7 | 42.9-44.8-53.4) | 4.4-7.7-23.1) |
| D2 | INV-21_P1 | INV-4_P3 | 91.4, −1.2, 8.4 | 0.59 | 19.2 | 22.2 | — | — |
| D2 | INV-21_P1 | INV-5_P3 | — | — | — | — | — | — |
| D2 | INV-21_P1 | INV-6_P3 | 90.6, −1.2, 8.7 | 0.53 | 17 | 24.1 | — | — |
| D4 | INV-8_P1 | INV-7_P3 | — | — | — | — | — | — |
| D6 | INV-9_P1 | INV-8_P3 | — | — | — | — | — | — |
| D7 | INV-10_P1 | INV-9_P3 | 93.4, −0.7, 8.9 | 0.67 | 14.9 | 17.3 | 31.3-34.2-39.4) | — |
| D12 | INV-13_P1 | INV-10_P3 | 88.1, −1.2, 9.1 | 0.52 | 20.6 | 25.5 | 39.6-42.8-53.3) | 4.9-9.3-23.6) |
| D14 | INV-11_P1 | INV-11_P3 | 91.3, −0.7, 6.2 | 0.28 | 9.5 | 38 | 60.4-62.1-66.8) | 38.6-41.0-48.1) |
| D16 | — | INV-12_P3 | — | — | — | — | 7-15-30) | — |
| D17 | — | INV-13_P3 | — | — | — | — | 9-17-32) | — |
| D18 | — | INV-14_P3 | — | — | — | — | 9-17-33) | — |
| D19 | — | INV-15_P3 | — | — | — | — | 10-19-37 | — |
| D20 | — | INV-16_P3 | — | — | — | — | 11-19-36 | — |
| D21 | — | INV-17_P3 | — | — | — | — | 43.4-47.6-58.6) | 14.7-20.1-38.2) |
| D22 | — | INV-18_P3 | — | — | — | — | 30.3-34.7-45.1) | 0.49-1.2-9.5) |
| D10 | — | INV-19_P3 | — | — | — | — | — | — |
| D9 | — | INV-20_P3 | 95.3, −0.6, 4.2 | 0.23 | 14 | 43.9 | 64.6-66-69.7) | 40.2-42.7-49.9) |
| D8 | — | INV-21_P3 | 87, 1.8, 15.3 | 0.71 | 21.2 | 17.3 | 28.6-32.1-39.8) | — |
| D13 | — | INV-22_P3 | 92.2, −0.8, 5.4 | 0.31 | 18.3 | 37 | 52.9-56.3-64.3) | 22.1-25.7-35.4) |
| D15 | — | INV-23_P3 | 88.1, −0.8, 8.3 | 0.44 | 20.7 | 28.9 | 45.8-49.5-59.8) | 15.8-20.8-36.3) |
| D11 | — | INV-24_P3 | 88.3, −1.2, 8.8 | 0.50 | 16.8 | 25.2 | 39.8-42.5-49.7) | — |
| D2 | INV-20_P1 | CE21 | — | — | — | — | 0-0-0 | 0-0-0 |

OP: opalescence
TP: translucency parameter
CR: contrast ratio

The contrast ratio corresponds to the “contrast ratio”-CR, determined from the luminance values (Y) of body P3 or P3z, measured according to the calorimetric reference system CIE 1931 (described in the standard ISO 11664-1), when the body is placed in front of a white background (Yw) or a black background (Yb), according to the following equation:

CR=Yb/Yw

The contrast ratio is a measure of the opacity of the body. In the field of dentistry, it is often used to determine the “translucency” according to the following equation:

translucency=1−CR.

The translucency parameter corresponds to the “translucency parameter”-TP of bodies P3 or P3z. The TP is determined by the difference between the colour measured in reflection mode when the body is placed in front of a white background (indices W) and the colour measured in reflection mode when the body is placed in front of a black background (indices B). The TP is calculated using the L*, a *, b* colour coordinates defined above, according to the following equation:

TP=[(L*_{W}−L*_{B})²+(a*_{W}−a*_{B})²+(b*_{W}−b*_{B})²]^{1/2}

As already indicated, “RIT” designates the value of direct transmittance (real in-line transmittance) while “TFT” designates the total transmittance (total-forward transmittance). These values are measured at ambient temperature, for a thickness of 1 mm.

All the examples according to the invention reported have optimum densification during step (f1). The method used makes it possible to obtain mechanical strengths of more than the materials of the prior art formed by a single component (doped zirconia) and of similar composition.

In Examples INV-9_P3 and INV-21_P3, resistances of more than 2.5 GPa and 2 GPa are obtained, respectively.

The optical properties of the materials obtained are superior to those of materials based on zirconia having a larger grain size (non-nanometric microstructures).

In terms of identical doping in yttria, the transmittance decreases when the value b* increases. According to the examples having a b* value of more than 2, transmittance is less than the maximum value indicated in Table 3 because of the presence of dyes which have been added to increase the b* value to obtain a colour which approximates the natural colour of dental enamel.

In Example INV-22_P3, good optical properties and an acceptable strength for dental applications are obtained.

Example INV-1_P3 has the best transmittance results for a composition of 3.35 mol % yttria, and a mechanical strength close to 2 Gpa.

Example INV-11_P3 has a high transmittance which is certainly unattainable for a zirconia-based material prepared according to a method which differs from that according to the invention and which is obtained by conventional sintering and with a grain size of less than 200 nm. Furthermore, this material has a mechanical strength of more than 650 MPa, allowing it to be used in non-dental applications.

9.2/Ceramic bodies were prepared from pre-ceramic bodies, with an intermediate pre-sintering step (e1).

TABLE 15
Ceramic bodies prepared after an intermediate pre-sintering step (e1)

P3

| | | | | | | Dureté | |
|---|---|---|---|---|---|---|---|
| | | | | (f1) | | Vickers | Rés. |
| Disp. | P1 | P2 | P3 | Frittage | Densité | (GPa) | méc. |
| D2 | INV-4_P1 | INV-13_P2 | INV-25_P3 | 2 h à 1150° C. | 99.8 | 13.3 | 1980 |
| D3 | INV-7_PI | INV-6_P2 | INV-26_P3 | 2 h à 1150° C. | 99.9 | 12.8 | 1350 |

| | P3 | | | | | | |
|---|---|---|---|---|---|---|---|
| | | | | | | TFT 555 nm- | RIT 555 nm- |
| | Taille de | | | | | 600 nm- | 600 mm- |
| P3 | grain (nm) | L*, a*, b* | CR | OP | TP | 780 nm | 780 nm |
| INV-25_P3 | 131 | — | — | — | — | — | — |
| INV-26_P3 | 90 | 92.2, −1.4, 4.2 | 0.55 | 18.2 | 23.1 | 41.9-44.7-54.7 | 4.9-8.5-24.1 |

The total duration of the heat treatment applied in step (f1), e.g., INV-25_P3 and INV-26_P3 is between 7.5 hours and 10 hours, with a plateau at 1150° C. for 2 hours.

10/Preparing Colour Bodies or Having a Colour Gradient or a Composition Gradient

10.1/Preparing a Body Having a Colour Gradient: Example A

Two dispersions of iron oxide nanoparticles Fe₃O₄ at basic pH are prepared as follows:

In a beaker, 7.9 g FeCl₂*4H₂O are dissolved in a solution containing 6.3 g HCl (1.5 M in water) and 36.3 g H₂O. The resulting solution is introduced into a solution containing 21.4 g of FeCl₃*6H₂O dissolved in 875 g of water. 75 ml of ammonia (8.6 M in water) are added at ambient temperature and by stirring vigorously to allow the coprecipitation of the FeII and FeIII ions and then the formation of magnetite Fe₃O₄ nanoparticles. The nanoparticles obtained, having a diameter of 8 nm (TEM), are collected by means of a magnet and peptised in 200 ml of an acid solution (2M HNO₃ in water). After stirring for 15 minutes, they are collected again by means of a magnet and redispersed in 500 ml of water, giving an acidic dispersion. The particles have a hydrodynamic diameter of less than 20 nm (DLS).

To disperse the nanoparticles at basic pH, the dispersion is separated into two parts:
M1: the nanoparticles are collected by means of a magnet and the supernatant is removed. The nanoparticles are redispersed in an aqueous solution containing 1 g of citric acid per g of magnetite formed. After flocculating, the particles are collected by means of a magnet. The supernatant is removed. The particles are then redispersed in 100 ml of water basified with 1 ml of NH₄OH (8.6 M in water). The resulting dispersion M1 contains 2.83% by weight of magnetite particles.
M2: the nanoparticles are collected by means of a magnet and the supernatant is removed. The nanoparticles are dispersed in an aqueous solution containing 1.5 g of TMAOH (tetramethylammonium hydroxide) per g of magnetite. The solution is stirred for 18 hours. Then, the nanoparticles are collected by means of a magnet and dispersed in 100 ml of water. The resulting dispersion M2 contains 2.27% by weight of magnetite particles.

The two dispersions M1 and M2 are very dark brown in colour.

400 ppm (by weight) of dispersion M1, measured in ppm of iron oxide equivalent Fe₂O₃, are introduced into 18 ml of the dispersion in Example INV-15_P1, the addition being performed drop by drop through stirring. Stirring was then maintained for 15 minutes. The dispersion changes to a cream colour after adding the M1 dispersion.

From the dispersion obtained, a green body 16 mm in diameter and 13 mm thick is obtained according to steps (b1) to (c1) in Example INV-15_P1. Throughout the filtration, a magnet (20 mm in diameter and 10 mm thick) is placed at a distance of 43 mm below the dispersion/filtration support interface, in a vertical filtration system in which the filtration is performed from top to bottom and the filtered solvent is discharged downwards. The magnet used is a Neodymium-Fer-Boron type magnet of quality 42 having a residual magnetic flux density (Br) of between 12900 Gauss to 13200 Gauss, a coercive field bHc of between 10.8 kOe and 12.0 kOe and an overall energy density of 40 MGOe to 42 MGOe. After step (c1), a green body of light brown colour free of cracks is obtained, then a plate 2 mm thick is cut from the green body, in the direction of filtration, by milling. Two pieces 2 mm thick are also obtained from the upper and lower parts of the green body. The 3 plates are then heat-treated with a debinding/sintering treatment according to step (f1) in Example INV-1_P3, and the colour of the ceramic bodies obtained is compared. After reducing the thickness to 1 mm and after polishing, the three pieces are the same very pale-yellow colour. A colour gradient is not formed.

10.2/Preparing a Body Having a Colour Gradient: Example B

400 ppm (by weight) of dispersion M1 and 400 ppm of dispersion M2 are introduced into 18 ml of dispersion in Example INV-15_P1, similar to Example A. The dispersion then has a homogeneous brown colour. The distance between the magnet and dispersion/filtration support interface is maintained at 43 mm. The green body obtained is then cut out and the pieces are heat-treated as in Example A. The three pieces have the same pale-yellow colour, which is clearly darker than in Example A. No colour gradient is formed. The transmittance is lower than in Example A.

10.3/Preparing a Body Having a Colour Gradient: Example C

800 ppm (by weight) of the M1 dispersion are introduced into 18 ml of the dispersion in Example INV-15_P1, similarly to Example A. The dispersion then changes to a brown colour. The distance between the magnet and the dispersion/filtration support interface is, this time, reduced to 5 mm during filtration. The green body obtained is then cut and the pieces are heat-treated as in Example A. The resulting piece of the upper part has a white colour as for a piece without the addition of dye, the resulting piece of the lower part has a yellow colour that is clearly darker than Example B. On the piece cut in the vertical direction, a colour gradient is visible in the first 5 mm of the piece from the lower part. A colour gradient thus formed over a thickness of 5 mm of the green body.

10.4/Preparing a Body Having a Colour Gradient: Example D

800 ppm (by weight) of dispersion M1 and 400 ppm of dispersion M2 are introduced into 18 ml of dispersion in Example INV-15_P1, similar to Example A. The changes to a homogeneous brown colour. The distance between the magnet and the dispersion/filtration support interface is maintained at 5 mm during filtration as in Example C. The green body obtained is then cut out and the pieces are heat treated as in Example A. The piece resulting from the upper pail has a very pale-yellow colour as in Example A. The piece resulting from the lower part has a yellow colour as in the lower part of Example C. On the piece cut out in the vertical direction, a colour gradient is visible throughout the piece. A colour gradient thus formed over the entire thickness of the green body.

TABLE 16
Characteristics of Examples A to D

| | MI | M2 | Distance magnet- | Formation of |
|---|---|---|---|---|
| Example | (PPrn) | (PPin) | filtration interface | a gradient |
| A | 400 | 0 | 43 | no |
| B | 400 | 400 | 43 | no |
| C | 800 | 0 | 5 | Yes over 5 mm |
| | | | | of thickness |
| D | 800 | 500 | 5 | Yes, over all |
| | | | | the thickness |

1. A method for preparing pre-ceramic body P2, comprising the following steps:
(a1) providing a dispersion of crystalline metal oxide particles having a mean size of 40 nm or less;
(b1) introducing the dispersion into a mould and forming a wet body by pressure filtration of the dispersion in the mould;
(b1′) optionally, pressing the wet body by pressure filtration;
(b2) demoulding the wet body under conditions of relative humidity of more than 80%;
(b3) optionally, when the wet body has a density gradient, removing the part of the wet body at the end of filtration;
(c1) forming a green body P1 by drying the wet body under conditions of relative humidity of more than or equal to 90%;
(c1′) optionally, shaping the green body P1;
(d1) optionally, debinding the green body; and
(e1) forming a pre-ceramic body P2 by pre-sintering green body P1 of one of steps (c1), (c1′) or
(d1), at a temperature of between 400° C. and 800° C.

2. The method according to claim 1, wherein the crystalline metal oxide particles in step (a1) are ZrO₂ particles having a mean size of between 3 nm and 25 nm,
the dispersion in step (a1) comprises 0.4 to 1.5% by weight of binder,
the pressure in step (b1) is between 5 bar and 50 bar, and
the green body P1 in step (c1) has a thickness of at least 5 mm.

3. The method according to claim 1 wherein the method comprises:
one step (b1′) of pressing the wet body by pressure filtration, and
one step (b3) consisting of removing the part of the wet body at the end of filtration when the wet body has a density gradient.

4. The method according to claim 1 wherein the method comprises:
one step (c1′) of shaping the green body P1, and
one step (d1) of unbinding the green body.

5. The method according to claim 1
wherein the method comprises:
one step (b1′) of pressing the wet body by pressure filtration,
one step (b3) consisting of removing the part of the wet body at the end of filtration when the wet body has a density gradient, and
one step (c1′) of shaping the green body P1.

6. The method according to claim 1 wherein the method comprises:
one step (b1′) of pressing the wet body by pressure filtration,
one step (c1′) of shaping the green body P1, and
one step (d1) of unbinding the green body.

7. The method according to claim 1 wherein the method comprises:
one step (b1′) of pressing the wet body by pressure filtration,
one step (b3) consisting of removing the part of the wet body at the end of filtration when the wet body has a density gradient, and
one step (d1) of unbinding the green body.

8. The method according to claim 1 wherein the method comprises:
one step (b1′) of pressing the wet body by pressure filtration,
one step (b3) consisting of removing the part of the wet body at the end of filtration when the wet body has a density gradient,
one step (c1′) of shaping the green body P1, and
one step (d1) of debinding the green body.

9. The method according to claim 1 wherein the method comprises:
one step (b1′) of pressing the wet body by pressure filtration, and
one step (d1) of debinding the green body.

10. A pre-ceramic body P2z of zirconium oxide having:
a mean grain size of less than 45 nm,
a density of between 52% and 68%, relative to the theoretical density in the absence of pores,
a hardness of more than 150 HV,
a mechanical biaxial bending strength of at least 25 MPa, and
a mean pore size of between 2 nm and 15 nm.

11. The pre-ceramic body according to claim 10, wherein pre-ceramic body P2z has a thickness of more than or equal to 5 mm and wherein the zirconium oxide is doped with 1 mol % to 15 mol % of a dopant selected from yttrium oxide, cerium oxide or a mixture of these two oxides.

12. (canceled)

13. A method for the preparation of a ceramic body P3, comprising the following steps:
(a1) providing a dispersion of crystalline metal oxide particles having a mean size of 40 nm or less;
(b1) introducing the dispersion into a mould and forming a wet body by pressure filtering the dispersion into the mould;
(b1′) optionally, pressing the wet body, by pressure filtration;
(b2) demoulding the wet body under conditions of relative humidity of more than 80%;
(b3) optionally, when the wet body has a density gradient, removing the part of the wet body at the end of filtration;
(c1) forming a green body P1 by drying the wet body under conditions of relative humidity higher than 90%;
(c1′) optionally, shaping the green body P1;
(d1) optionally, unbinding the green body P1;
(e1) optionally, forming a pre-ceramic body P2 by pre-sintering green body P1 of one of steps (c1), (c1′) or (d1), at a temperature between 400° C. and 800° C.;
(e2) optionally, shaping pre-ceramic body P2; and
(f1) forming a ceramic body P3 by sintering green body P1 of one of steps (c1), (c1′) or (d1) or by sintering pre-ceramic body P2 of step (e1) or (e2), sintering being performed at a temperature of between 900° C. and 1300° C.

14. A method for the preparation of a green body, comprising the following steps:
(a1) providing a dispersion of crystalline metal oxide particles having a mean size of 40 nm or less;
(b1) introducing the dispersion into a mould and forming a wet body by pressure filtering the dispersion into the mould;
(b1′) optionally, pressing the wet body by pressure filtration;
(b2) unmoulding the wet body under conditions of relative humidity of more than 80%;
(b3) optionally, when the wet body has a density gradient, removing the part of the wet body at the end of filtration;
(c1) forming a green body P1 by drying the wet body under conditions of relative humidity higher than 90%, and
(c1′) optionally, shaping the green body P1.

15. The method according to claim 13
wherein the dispersion in step (a1) comprises a binder and a dispersing agent,
and in that the pressurised filtration in step (b1) is performed by applying a pressure of between 5 bar and 50 bar, and wherein the crystalline metal oxide particles are zirconium oxide particles, doped with 1 mol % to 15 mol % of a dopant chosen from Yttrium oxide, cerim oxide or a mixture of these two oxides.

16. (canceled)

17. A ceramic body P3z of crystalline zirconium oxide having:
a mean grain size of less than 200 nm,
a density of more than 99%,
a mechanical biaxial bending strength of at least 600 MPa, and
a mean pore size of less than or equal to 20 nm.

18. The ceramic body P3 according to claim 17, wherein the zirconium oxide is doped with 1.5 mol % to 2.5 mol % of yttrium oxide and has a mean mechanical biaxial bending strength of at least 2000 Mpa and an opalescence of between 9 and 23.

19. (canceled)

20. The ceramic body P3z according to claim 17, wherein the ceramic body is based on zirconium oxide doped with 3.5 mol % to 6.5 mol % of yttrium oxide and has an opalescence of between 16 and 22.

21. The ceramic body P3z according to claim 17 wherein the ceramic body is based on zirconium oxide doped with at least 2.5 mol % yttria, and has a transmittance of at least 47% and a direct transmittance value of at least 22% at 780 nm, for a thickness of 1 mm.

22. (canceled)

23. A green body comprising crystalline zirconium oxide particles having a mean size of 3 nm to 25 nm, pores having a mean size of between 2 nm and 6 nm, and being free of cracks of more than 50 μm, having a density of between 45% and 60% relative to the theoretical density, and a thickness of more than or equal to 5 mm.

24. The pre-ceramic body P2z according to claim 10, wherein the pre-ceramic body P2z has a concentration gradient of a colouring agent or a colouring agent precursor, and a lanthanum oxide concentration of less than 0.1 mol %.

25. (canceled)

26. (canceled)
